SEQUENCE LISTING (18 sequences)

SEQ ID NO: 1 - DNA, 5 bp, Human
  misc_feature (1)..(5): n equals any nucleotide
tnttt

SEQ ID NO: 2 - DNA, 5 bp, Human
  misc_feature (1)..(5): n equals any nucleotide
tttnt

SEQ ID NO: 3 - DNA, 5 bp, Human
tttat

SEQ ID NO: 4 - DNA, 5 bp, Human
tttgt

SEQ ID NO: 5 - DNA, 5 bp, Artificial sequence
  Muted ATRS sequence
cctat

SEQ ID NO: 6 - DNA, 5 bp, Artificial Sequence
  Muted ATRS Sequence
ggtgt

SEQ ID NO: 7 - protein, 13 aa, Human
RRRHIVRKRTLRR

SEQ ID NO: 8 - DNA, 80 bp, Artificial Sequence
  Primer
gacgtctcgagaattcatcgnnnnnnnnnnnnnnnnnnnnnnnnnnnnnnnnnnnncgatggatccatccatgtcagact

SEQ ID NO: 9 - DNA, 20 bp, Artificial Sequence
  Primer
gacgtctcgagaattcatcg

SEQ ID NO: 10 - DNA, 24 bp, Artificial Sequence
  Primer
cgatggatccatccatgtcagact

SEQ ID NO: 11 - DNA, 23 bp, Artificial Sequence
  Primer
gaggggactaatatttccagcaa

SEQ ID NO: 12 - DNA, 21 bp, Artificial Sequence
  Primer
taaagggatttcagcttagca

SEQ ID NO: 13 - DNA, 4879 bp, Human
accaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtcttagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctcttt

SEQ ID NO: 14 - DNA, 364 bp, Mouse
tgctggtgttgctgaccgcgctctgcgcaggtggggcgttggaggaaaagaaagtctgccaaggcacaagtaacaggctcacccaactgggcacttttgaagaccactttctgagcctgcagaggatgtacaacaactgtgaagtggtccttgggaacttggaaattacctatgtgcaaaggaattacgacctttccttcttaaagaccatccaggaggtggccggctatgtcctcattgccctcaacaccgtggagagaatccctttggagaacctgcagatcatcaggggaaatgctctttatgaaaacacctatgccttagccatcctatccaactatgggacaaacagaactgggcttag

SEQ ID NO: 15 - DNA, 2643 bp, Human
gccccggcgccgccgccgcccagaccggacgacaggccacctcgtcggcgtccgcccgagtccccgcctcgccgccaacgccacaaccaccgcgcacggccccctgactccgtccagtattgatcgggagagccggagcgagctcttcggggagcagcgatgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatggaagctacatagtgtctcactttccaagatcattctacaagatgtcagtgcactgaaacatgcaggggcgtgttgagtgtggaaggatcttgacaagttgttttgaagatagcattttgctaagtccctgaggtcactggtcctcaaagcggcatggcgcatggcgtggctggttctgccacatgccagctgtgtgacctctgagactccacttcttccgtgctgaaaataaagaaggagttttactaaggaccaaacaagataatgaatgtgaaactgctccatgaaccccaaagaattatgcacatagatgcgatcattaagatgcgaagccatcgagttaccacctggcatgcttaaactgtaaagagtgggtcaaagtaaactgaattggaaaatccaaagttatgcagaaaaacaataaaggagatagtaaaaagggttaacgagccagtccaggggaagcgaagaagacaaaaagagtccttttctgggccaagtttgataaattaggcctcccgaccctttgctctgttgctttatcaactctactcggcaataacaat

SEQ ID NO: 16 - protein, 1342 aa, Human
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 17 - protein, 120 aa, Mouse
LVLLTALCAGGALEEKKVCQGTSNRLTQLGTFEDHFLSLQRMYNNCEVVLGNLEITYVQRNYDLSFLKTIQEVAGYVLIALNTVERIPLENLQIIRGNALYENTYALAILSNYGTNRTGL

SEQ ID NO: 18 - protein, 657 aa, Human
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGSYIVSHFPRSFYKMSVH

(19) United States
(12) Patent Application Publication
(10) Pub. No.: US 2003/0053995 A1
(43) Pub. Date: Mar. 20, 2003

(54) Methods and compositions for inhibiting EGF receptor activity

(76) Inventors: Mien-Chie Hung, Houston, TX (US); Shiaw-Yih Lin, Pearland, TX (US)

Correspondence Address: FULBRIGHT & JAWORSKI, LLP, 1301 MCKINNEY, SUITE 5100, HOUSTON, TX, 77010-3095, US

(21) Appl. No.: 10/172,620

(22) Filed: Jun. 14, 2002

Related U.S. Application Data
(60) Provisional application No. 60/298,579, filed on Jun. 15, 2001.

(51) Int. Cl. A61K 48/00

(52) U.S. Cl. 424/93.21; 514/44

(57) ABSTRACT

The present invention is directed to association of nuclear receptor tyrosine kinase, such as EGFR, with highly proliferative tissue following its translocation from the cell membrane. The nuclear localization of the receptor tyrosine kinase is affiliated with transcription activity, and the specific sequence associated with such activity, particularly for EGFR, is disclosed.

The present invention claims priority to U.S. Provisional Patent Application No. 60/298,579, filed Jun. 15, 2001, incorporated by reference herein in its entirety.

### FIELD OF THE INVENTION

The present invention is directed to the fields of cellular biology and molecular biology. In particular, the present invention is related to transcription factor activity of a receptor tyrosine kinase and the sequence to which it targets. More particularly, the present invention is directed to EGF receptor transcription activity and target sequences.

### BACKGROUND OF THE INVENTION

Epidermal growth factor receptor (EGFR) is a 170 kDa transmembrane glycoprotein which possesses the intrinsic tyrosine kinase activity (Cohen et al., 1982). EGFR exerts a great variety of biological functions including cell survival, mitogenic response, differentiation and cell motility (Khazaie et al., 1993). Many ligands for EGFR have been identified including epidermal growth factor (EGF), transforming growth factor α (TGF-α), amphiregulin (AR), epiregulin (EP), Batacellulin (BTC), Heparin-binding EGF-like growth factor (HB-EGF) and Schwannoma-derived growth factor (SDGF). The EGF-family of peptides is significantly involved in the regulation of mammary-gland development, morphogenesis and lactation, and also implicated in the pathogenesis of human breast cancer (Normanno and Ciardiello, 1997).

Studies for EGFR have been primarily focused on conventional signal transduction pathways, such as MAPK (Boonstra et al., 1995, PLCγ (Anderson et al., 1990) and P13K (Hu et al., 1992) However, it has long been known that many functions of EGFR, such as EGF-induced DNA synthesis and mitogenic effect required other mechanisms besides those early transient responses (Carpenter and Cohen, 1979; Knauer et al., 1984; Defize et al., 1986). In addition, Wakshull and Wharton have reported that stabilized complexes of EGF-EGFR on the cell surface were not able to induce DNA synthesis, although transient responses could be activated (Wakshull and Wharton, 1985). EGFR and its ligands have been repeatedly observed in the nucleus, such as in cell lines, human placenta, regenerating liver (Zimmermann et al., 1995) and in many different cancer types (Tervahauta et al., 1994; Kamio et al., 1990; Gusterson et al., 1985; Lipponen and Eskelinen, 1994). Thus, certain critical activities of EGRF signaling, such as the function of nuclear EGFR, remain unclear.

Marti and Wells (2000) identify nuclear accumulation of an EGFR that lacks the transmembrane domain. Although the variant accumulates in the nucleus of mouse 3T3 cells and mouse NR6 cells following incubation with EGF, the nuclear accumulation required the presence of wildtype EGFR. Furthermore, Marti et al. (2001) identify the presence of nuclear EGF and EGFR in the thyroid, particularly as corresponding to increased growth associated with the thyroid, as seen in Graves' disease, papillary carcinoma and follicular adenomas/carcinomas of the thyroid.

Xie and Hung (1994) show nuclear localization of the transmembrane p185^{neu} tyrosine kinase and transcriptional activation. Given that an inherent tyrosine kinase activity of this transmembrane tyrosine kinase was unknown, the authors fused a soluble region of p185^{neu} to the GAL4 DNA-binding domain in an artificial system and demonstrated transactivation of the reporter β-galactosidase expression. Furthermore, it was demonstrated that p185^{neu} localizes to the nucleus and has a greater extent of tyrosine phosphorylation compared to nonnuclear-localized forms. However, this reference contains no suggestion that native p₁₈₅^{neu} contains a DNA binding domain, as evidenced by the fact that the authors fused p185^{neu} to a non-native DNA binding domain.

### SUMMARY OF THE INVENTION

Although a receptor tyrosine kinase, such as EGFR, has been detected in the nucleus in many tissues and cell lines, the inventors demonstrate herein that nuclear EGFR correlates strongly with highly proliferating tissues. For example, when fused to GAL4 DNA binding domain, the C-terminus of EGFR acts as a strong transactivation domain. More importantly, the receptor complex binds and activates ATRS (AT-rich consensus sequence)-dependent transcription, including the ATRS in Cyclin D1 promoter. Using chromatin immunoprecipitation assays, the inventors further demonstrated that nuclear EGFR associated with promoter region of Cyclin D1 in vivo. Therefore, EGFR functions as a transcription factor to activate genes required for highly proliferating activities.

In accordance with the objects of the present invention, a receptor tyrosine kinase that resides in the cell membrane is assayed for transcriptional activity, such as in the case wherein the enzyme translocates to the nucleus. Furthermore, the sequence through which the transcriptional activity of the receptor tyrosine kinase acts is obtained. In one embodiment, a receptor tyrosine kinase is pre-localized to the nucleus with a ligand being trafficked there after binding to the cell surface receptor. In an alternative embodiment, the ligand and receptor translocate to the nucleus together, such as if one or both components comprise a nuclear localization signal. In another embodiment, the nuclear receptor tyrosine kinase is a splice variant of the transmembrane form of the receptor.

Also, in accordance with the objects of the present invention, a therapeutic effect is achieved through inhibiting at least partially the activity of EGFR in highly proliferating tissues. In a specific embodiment, the therapeutic effect is the treatment of cancer, a disease of abnormally high proliferation, such as breast cancer, glioblastoma, head and neck cancer, bladder cancer, pancreatic cancer, colon cancer, lung cancer, thyroid cancer, and/or brain cancer. In a specific embodiment, there is interference of the translocation of EGFR from the membrane to the nucleus. In another specific embodiment, the transactivation by EGFR of a target nucleic acid sequence is inhibited at least partially. In an additional specific embodiment, the ATRS sequence is utilized as a screening tool for antimitotic drugs. For example, the ATRS sequence is operably linked to a reporter sequence, and expression in the presence of a test compound is assayed, wherein a reduction in the expression indicates the test compound is useful for the treatment of undesired proliferation of cells in a tissue or tissues. In another embodiment of the present invention, the ATRS sequence is associated with a moiety for directed killing of a cell. In specific examples, the moiety is a toxin or a pro-apoptotic gene.

In an object of the present invention, there is a method of treating a cell having upregulated EGFR expression, such as for a proliferative disorder, comprising administering to the cell an EGFR-regulated promoter sequence. An EGFR-regulated promoter sequence is a sequence through which EGFR acts as a transcription factor, either directly or in directly, and in some embodiments is in a complex. Examples of EGFR-regulated promoter sequences include TNTTT (SEQ ID NO:1) and TTTNT (SEQ ID NO:2). The EGFR-regulated promoter sequences are operably linked to a therapeutic polynucleotide and may be present in multiple copies. In a specific embodiment, the EGFR-regulated promoter sequence is an AT-rich minimal sequence (ATRS). In a specific embodiment, the therapeutic polynucleotide is a tumor suppressor, tumor associated gene, growth factor, growth-factor receptor, signal transducer, hormone, cell cycle regulator, nuclear factor, transcription factor or apoptic factor. In another specific embodiment, the tumor suppressor is selected from the group consisting of Rb, p53, p16, p19, p21, p73, DCC, APC, NF-1, NF-2, PTEN, FHIT, C-CAM, E-cadherin, MEN-I, MEN-II, ZACI, VHL, FCC, MCC, PMS1, PMS2, MLH-1, MSH-2, DPC4, BRCA1, BRCA2 and WT-1. In a further specific embodiment, the growth-factor receptor is selected from the group consisting of FMS, ERBB/HER, ERBB-2/NEU/HER-2, ERBA, TGF-β receptor, PDGF receptor, MET, KIT and TRK. In an additional specific embodiment, the signal transducer is selected from the group consisting of SRC, AB1, RAS, AKT/PKB, RSK-1, RSK-2, RSK-3, RSK-B, PRAD, LCK and ATM. In an additional specific embodiment, the transcription factor or nuclear factor is selected from the group consisting of JUN, FOS, MYC, BRCA1, BRCA2, ERBA, ETS, EVII, MYB, HMGI-C, HMGI/LIM, SKI, VHL, WT1, CEBP-a, NFKB, IKB, GLI and REL. In a further specific embodiment, the growth factor is selected from the group consisting of SIS, HST, INT-1/WT1 and INT-2. In another specific embodiment, the apoptic factor is selected from the group consisting of Bax, Bak, Bim, Bik, Bid, Bad, Bcl-2, Harakiri, granzyme B and ICE proteases. In another specific embodiment, the tumor associated gene is selected from the group consisting of CEA, mucin, MAGE and GAGE. In another specific embodiment, the proliferative disorder is cancer. In a further specific embodiment, the cell is in vivo. In a further specific embodiment, the cell is in a human.

In an additional object of the present invention there is a method of screening for a modulator of an EGFR-regulated promoter sequence, comprising the steps of introducing to a cell a nucleic acid construct comprising a nucleic acid sequence of SEQ ID NO:1 or SEQ ID NO:2 operably linked to a reporter sequence; contacting the cell with a candidate modulator; and assaying for a change in expression of the reporter sequence. In a specific embodiment, the reporter sequence expression is upregulated. In another specific embodiment, the reporter sequence expression is downregulated. In a further specific embodiment, the reporter sequence is selected from the group consisting of luciferase, green fluorescent protein, blue fluorescent protein, β-galactosidase, and chloramphenicol acetyl transferase. In a specific embodiment, the candidate modulator is a protein, a small molecule, a nucleic acid molecule, an antisense molecule, a ribozyme, an antibody, or a combination thereof. In a further specific embodiment, the candidate modulator is determined to be a modulator of an EGFR-regulated promoter sequence. In an additional specific embodiment, the method further comprises administering to an individual with cancer a pharmaceutically acceptable formulation of said modulator.

In an additional object of the present invention there is a method for identifying transcription factor activity for a receptor tyrosine kinase, comprising the step of assaying the receptor tyrosine kinase for DNA binding activity. In a further specific embodiment, the method further comprises identifying the target DNA sequence of the DNA binding. In another specific embodiment, the receptor tyrosine kinase is selected from the group consisting of insulin receptor, nerve growth factor receptor, fibroblast growth factor receptor, platelet-derived growth factor receptor, growth hormone receptor, IL-1 receptor, HER/neu, interferon alpha receptor, interferon beta receptor, and interferon gamma receptor, IL-5 receptor, angiogenin receptor, erythropoietin receptor, and G-CSF (granulocyte colony stimulating factor) receptor. In an additional specific embodiment, the DNA binding activity of said receptor tyrosine kinase is direct. In a further specific embodiment, the DNA binding activity of the receptor tyrosine kinase is through an agent which binds the target directly.

In another object of the present invention, there is a method of treating cancer in an individual comprising the step of reducing translocation of a receptor tyrosine kinase from a membrane of a cancerous cell of the individual to the nucleus of said cell. In a specific embodiment, the receptor tyrosine kinase is EGFR.

In an additional object of the present invention, there is a method of treating cancer in an individual comprising the step of reducing transcription factor activity of a receptor tyrosine kinase in a cancerous cell of the individual. In a specific embodiment, the receptor tyrosine kinase is EGFR.

In an additional object of the present invention, there is as a composition of matter a pharmaceutical composition comprising a nucleic acid construct comprising a nucleic acid sequence of SEQ ID NO:1 or SEQ ID NO:2 operably linked to a therapeutic nucleic acid sequence; and a pharmaceutically acceptable carrier. In a specific embodiment, the therapeutic nucleic acid sequence is a tumor suppressor, tumor associated gene, growth factor, growth-factor receptor, signal transducer, hormone, cell cycle regulator, nuclear factor, transcription factor or apoptic factor.

In another embodiment of the present invention, there is a method of treating an individual with cancer comprising administering to said individual a modulator that affects EGFR transcriptional activity.

In an additional embodiment of the present invention, there is a method of identifying a cancerous cell in an individual, comprising identifying a nuclearly localized receptor tyrosine kinase in said cell. In a specific embodiment, the receptor tyrosine kinase is EGFR. In a specific embodiment, the cancerous cell is a breast cancer cell, glioblastoma cell, head and neck cancer cell, bladder cancer cell, pancreatic cancer cell, colon cancer cell, lung cancer cell, thyroid cancer cell, or brain cancer cell. In an additional specific embodiment, the method further comprises the step of treating said individual for said cancer. In a specific embodiment, the treating step comprises administering to the individual a pharmaceutically acceptable formulation of a nucleic acid sequence comprising an EGFR-regulated promoter sequence operably linked to a therapeutic polynucleotide. In an additional specific embodiment, the EGFR-regulated promoter sequence is SEQ ID NO:1 or SEQ ID NO:2. In a further specific embodiment, the treating step comprises administering to said individual a pharmaceutically acceptable formulation of a modulator that inhibits transcriptional activity of a receptor tyrosine kinase.

Other and further objects, features and advantages would be apparent and eventually more readily understood by reading the following specification and by reference to the company drawing forming a part thereof, or any examples of the presently preferred embodiments of the invention are given for the purpose of the disclosure.

### BRIEF DESCRIPTION OF THE FIGURES

FIG. 1 demonstrates immunohistochemical staining of different tissues for EGFR. In FIG. 1A, uterus from a pregnant mouse (top left) and a non-pregnant mouse (top right) is shown. The bottom panels show control experiments: 10× EGFR competitive peptide was added (bottom left); PBS was used instead of primary antibody (bottom middle), and 468 cell-derived tumors used as a positive staining control (bottom right). In FIG. 1B, normal human mouth mucosa ×200 (top left) and ×400 (top right) is shown. Bottom panels show the respective negative controls using antibody with 10× competitive peptide (bottom left) or PBS instead of primary antibody (bottom right). In FIG. 1C, human oral cancer sample is shown (×400 (top)). Bottom panels show negative and positive controls as described for panel a. (d) Cell lines were immunostained with EGFR antibody and analyzed by confocal microscopy. The yellow signals indicated the localization of EGFR in the nucleus.

FIG. 2 demonstrates detection of EGFR in the nuclear fractions of A431 and MDA-MB-468 cells. In FIG. 2A, the nuclear (80 μg) and non-nuclear (20 μg) fractions from cells treated with EGF (+) or without (−) were subjected to immunoprecipitation with monoclonal antibody against human EGFR or c-myc (control) and then blotted with anti-phosphotyrosine (PY20) antibody (top) or with sheep anti-human EGFR antibody (bottom). In FIG. 2B, nuclei were prepared from unstimulated and EGF-stimulated MDA-MB-468 cells (N⁻ and N⁺, respectively) and then mixed with the non-nuclear fraction from the cells stimulated with EGF for 30 minutes (S⁺). The nuclei were then separated from the S⁺ fraction, and the nuclear extract was isolated and analyzed by immunoblotting with anti-phosphotyrosine (PY20) antibody.

FIG. 3 shows a time-course study of EGFR nuclear localization. In FIG. 3A, A431 cells were stimulated with EGF and incubated at 37° C. for 1-30 minutes or at 37° C. for 1 minute and then 4° for another 14 minutes (lane 6) or 29 minutes (lane 7). Then, the nuclear extract (top) and non-nuclear fraction (middle) were subjected to western blotting with anti-EGFR and anti-phosphotyrosine (PY20) antibodies or with pRB antibody as the loading control. The results were then diagrammatically plotted as shown in the bottom panel. The density of the bands at time 0 were defined as 1 after subtracted with the background by using NIH Image software to quantify the signals. In FIG. 3B, specific cell surface labeling of EGFR by crosslinking with ¹²⁵I-EGF is shown. At top, ¹²⁵I-EGF-EGFR cross-linked proteins in the non-nuclear (lane1) and nuclear fractions (lane 2) are visualized by autoradiography. In the presence of the cold EGF, the level of cross-linking was reduced (lane 3 and 4). At bottom, crosslinking was as described above except that a non-membrane permeable cross-linker was used.

FIG. 4 shows activation of gene expression by C-terminus of EGFR (PRR domain). In FIG. 4A, relative CAT activity of the reporter gene was measured when different GAL4-EGFR expression constructs were cotransfected into NIH3T3 cells. In FIG. 4B, results of experiments similar to those described above are shown except that other different cell lines and a luciferase reporter were used. FIG. 4C shows dose-dependent transactivation in NIH 3T3 cells.

FIG. 5 demonstrates the AT rich consensus sequences (ATRS) identified by CASTing. At top, sequences of six identified clones with the ATRS marked in each are presented. At bottom, an EGFR-associated protein complex specifically bound to a DNA probe containing putative EGFR binding sites.

FIG. 6 shows EGF activation of ATRS-specific reporter gene expression in EGFR overexpressed cell lines. (FIG. 6A) A431, (FIG. 6B) MDA-MB-468, (FIG. 6C) HBL 100, and (FIG. 6D) CHO cells. All four lines were transfected with a luciferase vector containing four repeats of wild-type (WT) or mutated (MT) ATRS sequences, with (+) or without (−) EGF (100 ng/ml).

FIG. 7 demonstrates ATRS-dependent activation of cyclin D1 promoter by EGF and association of EGFR with cyclin D1 promoter in vivo. In FIG. 7A, MDA-MB-468 cells were transfected with a luciferase vector containing the cyclin D1 promoter with two intact ATRS or the same promoter with the ATRS mutated. After 24 hours of incubation, cells were treated with (+) or without (−) EGF (100 ng/ml). In FIG. 7B, A431 cells were treated with (+) or without (−) EGF (100 ng/ml) for 30 min, cross-linked with 1% HCHO, and nuclear lysate prepared. After precipitation with the EGFR antibodies (1 Santa Cruz, 2 NeoMarkers) or normal rabbit IgG, cyclin D1 promoter region was amplified by PCR. Input nuclear DNA (In) or water (dw) were used as PCR controls.

### DETAILED DESCRIPTION OF THE INVENTION

It will be readily apparent to one skilled in the art that various substitutions and modifications may be made in the invention disclosed herein without departing from the scope and spirit of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

The present invention addresses the heretofore unknown transcriptional activity of a transmembrane receptor tyrosine kinase. Although receptor tyrosine kinases have been demonstrated to localize nuclearly, their function in the nucleus had previously not been determined. As described herein, there is a correlation between the nuclearly-localized receptor tyrosine kinase EGFR and highly proliferative tissues. Furthermore, for the specific receptor tyrosine kinase EGFR, nuclear localization increases upon treatment with the ligand EGF, and following such the nuclear-localized EGFR is highly phoshorylated. Moreover, the C-terminus alone is capable of activating a reporter sequence in vitro in a dose-dependent manner. In the present invention, the inventors provide identification of a consensus sequence, ATRS, through which EGFR binds.

Thus, in accordance with the teachings provided herein, a receptor tyrosine kinase is assayed for the ability to activate transcription, such as by binding nucleic acid sequence, and its target sequence or sequences are identified. Furthermore, the specific ATRS target sequence for EGFR, described herein, is useful to facilitate treatment of a highly proliferating cell and/or tissue. For example, the ATRS sequence is operably linked to a therapeutic nucleic acid sequence and introduced into a cell. There is subsequently selective expression in the cancer cells in which the overexpress EGFR gene product act through the ATRS to express the therapeutic nucleic acid sequence. Thus, administration of the ATRS operably linked to a therapeutic nucleic acid sequence is primarily not deleterious in normal tissues since the EGFR gene product is not highly overexpressed in these tissues, and as a result side effects are reduced.

The ATRS sequence of the present invention, and any other target sequences analogously identified for a receptor tyrosine kinase, are used to screen for agents which affect expression through the sequence. For example, the ATRS sequence is operably linked to a reporter sequence, such as GFP or luciferase, and administered into a cell. Upon exposing the cell to candidate agents, the reporter expression is assayed for each candidate. The agent which increases expression of the reporter sequence would similarly affect expression of the endogenous ATRS-regulated nucleic acid sequence. In a similar fashion, a candidate agent which reduces expression of the reporter sequence would similarly affect expression of the endogenous ATRS-regulated nucleic acid sequence and would be useful in the treatment of a highly proliferative tissue such as cancer.

As used herein, the term “target” refers to a first nucleic acid sequence through which an agent of interest affects expression of a second nucleic acid sequence. In one embodiment, the target is bound directly by the agent of interest. In another embodiment, the target is bound by a complex comprising the agent of interest. In an additional embodiment, the first nucleic acid sequence and the second nucleic acid sequence are configured in cis. In a preferred embodiment, the agent of interest is a receptor tyrosine kinase. In an additional preferred embodiment, the receptor tyrosine kinase is nuclearly localized. In a further preferred embodiment, the receptor tyrosine kinase is EGFR.

A skilled artisan recognizes that nucleic acid sequences and/or amino acid sequences useful to the present invention are readily obtainable through publicly available databases, such as the National Center for Biotechnology Information's GenBank database or commercially available databases, such as from Celera Genomics, Inc. (Rockville, Md.). For example, representative EGFR nucleic acid sequences, followed by their GenBank Accession Nos., include SEQ ID NO:13 (M29366); SEQ ID NO:14 (L06864); and SEQ ID NO:15 (K03193). Also, for example, representative EGFR amino acid sequences, followed by their GenBank Accession Nos., include SEQ ID NO:16 (AAA35790); SEQ ID NO:17 (AAA53029); and SEQ ID NO:18 (AAA52371). Other sequences, such as additional receptor tyrosine kinases, are similarly readily available. Furthermore, a skilled artisan recognizes that once a target sequence has been identified, such as by methods described in the Examples provided herein, the target sequence may be used to screen the databases for identical or similar sequences among other nucleic acid sequences.

A skilled artisan recognizes that many ligands for EGFR have been identified, including epidermal growth factor (EGF), transforming growth factor α (TGF-α), amphiregulin (AR), epiregulin (EP), Batacellulin (BTC), Heparin-binding EGF-like growth factor (HB-EGF) and Schwannoma-derived growth factor (SDGF). Additional ligands for EGFR are included in the scope of the invention, and a skilled artisan is aware of a variety of molecular biology tools and reagents to routinely determine them.

A skilled artisan also recognizes that transmembrane receptors have been located in the nucleus with no clear identification of the functions, such as insulin (Vigneri et al., 1978), nerve growth factor (Rakowicz-Szulczynska et al., 1986, 1988), fibroblast growth factor (Maher, 1996; Stachowiak et al., 1996) platelet-derived growth factor (Rakowicz-Szulczynska et al., 1986), growth hormone (Lobie et al., 1994), IL-1 (Curtis et al., 1990) HER/neu (Xie and Hung, 1994; Cohen et al., 1992), interferon alpha receptor, interferon beta receptor, and interferon gamma receptor, IL-5 receptor, angiogenin receptor, erythropoietin receptor, and G-CSF (granulocyte colony stimulating factor) receptor. These transmembrane receptors and others not listed herein are easily screened for transcriptional activity in accordance with the teachings provided herein.

In accordance with the teachings provided herein, a skilled artisan recognizes that multiple downstream targets for EGFR or another receptor tyrosine kinase, which comprise the appropriate target sequence, such as ATRS, are analogously identified. Examples of downstream targets besides Cyclin D1 that comprise an ATRS sequence include myc and JunB.

I. EGFR in Normal Development

EGFR is expressed throughout the developmental process and in a variety of undifferentiated and differentiated cells (Gospodarowicz, 1981). EGFR and one of its ligands, TGF-α, are expressed in the preimplantation conceptus and play a role in blastocoel expansion, embryo-uterine signaling, and the implantation process (Dardik and Schultz, 1991; Arnholdt et al., 1991; Zhang et al., 1992). Among the functions attributed to EGFR activity are the proliferation and development of specific epithelial territories in the embryo, including branch point morphogenesis and maturation of early embryonic lung tissue, skin development, and promoting survival of early progenitor cells of cleft palate (Abbott and Pratt, 1991; Warburton, 1992).

An interplay of the actions of EGFR and estrogen receptor has been proposed to be required for the differentiation of normal mammary epithelial cell as well as the induction of uterine and vaginal growth (Nelson et al., 1991; Ignar-Trowbridge et al. 1992). EGFR expression is high in the cap-cell layer of the terminal end buds (Daniel, 1987), a proliferating cell population (Coleman, 1988) that is presumed to be the stem cell population of both the luminal and myoepithelial cells of mammary ducts (Daniel and Siberstein, 1987). The cap cell layer is devoid of estrogen receptors which instead are abundant in the surrounding stromal cells (Daniel et al., 1987). It has been proposed that estrogen may regulate the growth of cap-cells through a paracrine mechanism by stimulating the production of EGF or TGF-α. In ovariectomized mice, the exogenous delivery of either EGF or TGF-α was sufficient to restore the pattern of normal ductal growth in the involuted mammary gland. In normal mice, distinctly different patterns of immunolocalization were observed for EGF (inner layers of terminal end buds and in ductal cells of mammary epithelium) and TGF-α (epithelial cap cell layer of the advancing terminal end bud and in stromal fibroblasts at the base of the terminal end bud) suggesting that each ligand plays a different role in normal mammary gland morphogenesis (Snedeker et al., 1991).

II. Role of EGFR in Malignant Development

Expression and activity of EGFR have been linked with a number of pre-malignant or malignant disease. Many types of epithelial malignancies display increased EGFR expression, including breast cancer (Harris et al., 1989; Sainsbury et al., 1985a), lung cancer (Hendler and Ozanne, 1984; Hendler et al., 1989; Veale et al., 1987), glioblastoma (Humphrey et al., 1988; Libermann et al., 1985), head and neck cancer (Eisbruch et al., 1987), and bladder cancer (Lipponen and Eskelinen, 1994; Neal et al., 1985), etc. In addition, overexpression of EGFR has been reported to correlate with a poor clinical outcome in many malignancies, such as cancers of the bladder (Harris et al., 1989; Neal et al., 1985), breast (Harris et al., 1989; Sainsbury et al., 1985a; Hawkins et al., 1991) and lung (Hendler et al., 1989; Veale et al., 1987). The overexpression of EGFR in the breast carcinoam is about 14% to 42%, depending on the groups conducting the studies (Hoskins and Weber, 1995).

When the oncogenic activities were studied, it was found that overexpression of EGFR was well correlated to the growth stimulation of culture cells under 3-dimensional culture condition (Minke et al., 1991), anchorage-independent environment (Lee et al., 1987), xenotransplants in immune deficient mice (Santon et al., 1986) (Filmus et al., 1987), and tumor progression and metastasis (Lichtner et al., 1988). Therefore, it is strongly believed that the aberrant expression and activation of EGFR is implicated in those transformation phenotypes and affect the prognosis and the response to therapy in clinic. The present invention and related technologies are directed to understanding more about receptor-dependent oncogenesis and improving the therapeutic outcomes for these types of cancers, such as by identifying downstream targets, biological functions and pathological effects of EGFR.

III. Signaling through the EGFR

EGFR is a prototype of a receptor tyrosine kinase. Activation of the EGFR begins by ligand binding to the extracellular domain, subsequently inducing a conformational change in the receptor and resulting in receptor dimerization. This oligomerization induces the dimerized receptor to cross-phosphorylate the C-terminal tail of its dimerization partner (Honegger et al., 1989; Kashles et al., 1988). The phosphorylated tyrosine residues in the C-terminus can then act as docking sites for proteins with SH2 domains (Pawson and Gish, 1993).

SH2 domains are regions of homology to the oncogene src and mediate protein-protein interactions by facilitating binding to phosphorylated tyrosine residues (Marais et al., 1995). Whereas the presence of phosphotyrosine creates a binding site for a protein containing an SH2 domain, the specificity of the protein binding is conferred by the amino acids that surround the tyrosine on the target protein (Pawson and Gish, 1992).

Two proteins immediately responsible for the transduction of the EGFR signal are Grb2 and Shc. Grb2 is a member of a family of proteins initially cloned based on the ability to bind phosphotyrosine residues (Skolnik et al., 1991). Grb2 is a 23-Kd protein that possesses no intrinsic enzymatic activity and consists almost entirely of a central SH2 domain flanked by two SH3 domains (Lowenstein et al., 1992). Both SH2 and SH3 domains are involved in protein-protein interactions; SH2 domains bind phosphotyrosine, whereas SH3 domains mediate the binding to proline-rich sequence (Pawson and Gish, 1992). The shc gene, which was cloned using an SH2 domain as probe, encodes three overlapping proteins of 46, 52, and 66 KDa that contain a single SH2 domain and, like Grb2, possess no enzymatic activity (Pelicci et al., 1992). Shc proteins can also interact with tyrosine-phosphorylated EGFR molecules via their SH2 domain (Batzer et al., 1995).

In a resting cell, Grb2 is found in the cytoplasm in a complex with the human homolog of Drosophila Son of Sevenless gene, Sos (Li et al., 1993). Tyrosine phosphorylation of the cytoplasmic tail of EGFR as well as other receptor tyrosine kinases creates docking sites for SH2-containing proteins and promotes the recruitment of both Shc and Grb2-Sos to the plasma membrane where the Shc proteins are substrates for EGFR kinase activity (Okada et al., 1995; Pelicci et al., 1992). Although both Shc and Grb2 contain SH2 domains and are therefore capable of binding EGFR, the predominant interaction in EGF-stimulated cells is between EGFR and Shc, and the interaction of Grb2-Sos with EGFR occurs indirectly through the binding of the Grb2 SH2 domain to phosphorylated tyrosines on Shc (Sasaoka et al., 1994).

The conventional signal transduction pathways for EGFR are activated very quickly and transiently upon the treatment of EGF. However, many biological functions including mitogenic effect of EGFR are not explained solely by those transient signals. Evidence has been obtained that EGF-induced DNA synthesis and mitogenic effects required other mechanisms besides those early responses. For example, it has long been known that in order to achieve DNA synthesis, EGF has to be retained in the culture medium for several hours. Removing the growth factor within an hour of treatment, in which all known early responses have been induced and completed, results in prevention of the DNA synthesis and the mitogenic activity (Carpenter and Cohen, 1979; Knauer et al., 1984). In addition, although the tyrosine kinase activity, which is required for all the known signal transduction pathways by EGFR, has a critical role in the cellular response to EGF, the induction of tyrosine kinase activity of the receptor is not sufficient to stimulate DNA synthesis (Defize et al., 1986). Consistent with this observation, others have also reported that stabilized complexes of EGF-EGFR on the cell surface were not able to induce DNA synthesis (Wakshull and Wharton, 1985). All of those studies strongly argue that EGFR plays other important roles within the cells (cytoplasmic or nuclear) to exert some of its biological functions, including mitogenic effects.

IV. EGFR and other Transmembrane Receptors in Nucleus

Since the known conventional signal transduction pathways mediated by EGFR fail to explain all of the biological functions of EGFR, as discussed above, other mechanisms must mediate those functions. The role of transmembrane receptors in signaling is traditionally viewed as being exclusively at the level of the membrane, whereby the receptor transfers the signal represented by ligand binding from the external cell surface, across the membrane, to within the cell through a variety of soluble cytosolic components which forward the signal on to the nucleus. The tenet of this view, however, is that there are additional mechanisms in the context of particular growth factors and cytokines by which the signals can reach the nucleus, whereby internalized ligands and receptors themselves translocate to the nucleus to modulate gene transcription (Jans, 1994). Many transmembrane receptors and their cognate ligands were found in the nucleus (Jans and Hassan, 1998), including EGFR and the receptors for insulin (Vigneri et al., 1978), nerve growth factor (Rakowicz-Szulczynska et al., 1986, 1988), fibroblast growth factor (Maher, 1996; Stachowiak et al., 1996) platelet-derived growth factor (Rakowicz-Szulczynska et al., 1986), growth hormone (Lobie et al., 1994), IL-1 (Curtis et al., 1990) and HER/neu (Xie and Hung, 1994; Cohen et al., 1992). Significantly, ligand/receptor endocytosis appears to be required to elicit full response of ligands and consequently, the biological functions of most of those receptors. The implication is that ligand-receptor internalization may be an important initial step of a pathway to target ligands and/or activated receptor to intracellular sites such as the nucleus and/or nuclear envelope.

EGFR and its ligands have been repeatedly observed in the nucleus, including in a variety of cell lines (Rakowicz-Szulczynska et al, 1986; Marti et al, 1991; Holt et al., 1994), human placenta (Cao et al, 1995), regenerating liver (Zimmermann et al., 1995) and in many different cancer types (Tervahauta et al., 1994; Kamio et al., 1990; Gusterson et al., 1985; Lipponen and Eskelinen, 1994). In a study of bladder cancer, Lipponen and Eskelinen found that EGFR was overexpressed in 35% of cases and distinct nuclear localization of EGFR was found in 31% of tumors (Lipponen and Eskelinen, 1994). Interestingly, the nuclear expression of EGFR in this study was found to correlated significantly to grade and mitotic frequency.

The function of nuclear localized EGFR is far from clear, but based on the literature a significant correlation exists between the nuclear localization of the receptor and the highly proliferative activity of the tissues. Consistent with the fact that the mitogenic effect of EGFR requires pathways other than the conventional transduction pathways, it is reasonable to suspect a role of nuclear EGFR in regulating the genes whose products are important for cell proliferation.

A nuclear localization signal in the EGFR resides in amino acid residues 645-657 of the cytoplasmic domain (Holt et al., 1994). Furthermore, it was shown that EGF caused an increase in the number of EGFR present in the nucleus and also an increase in the phosphotyrosine content in the nucleus. In addition, it has been demonstrated that Schwannoma-derived growth factor, which belongs to the EGF family, contains two basic amino acid clusters that are homologous to the nuclear localization signal of simian virus 40-encoded large tumor antigen (Kimura, 1993). Deletion of these nuclear localization signals from the growth factor resulted in a loss of the mitogenic effect, but the early responses, such as activation of the early genes NGFI-A and c-fos, were unimpaired. A similar observation was also found for amphiregulin, another ligand for EGFR (Johnson et al., 1991). Elucidation of the functions of EGFR and its ligands in the nucleus is crucial to understand the mitogenic effect of EGFR signaling and its implication when overexpressed in cancers, such as breast cancer.

V. Screening for Modulators of ATRS-Regulated Expression

A skilled artisan recognizes, based on the examples and teachings provided herein, that analogous methods and compositions are useful upon target identification with receptor tyrosine kinases other than EGFR. In a specific embodiment, the present invention further comprises methods for identifying modulators of the function of expression regulated by an ATRS sequence (SEQ ID NO:1 or SEQ ID NO:2). These assays may comprise random screening of large libraries of candidate substances; alternatively, the assays may be used to focus on particular classes of compounds selected with an eye towards structural attributes that are believed to make them more likely to modulate the function of ATRS-regulated expression.

By function, it is meant that one may assay for the activity for ATRS sequence to regulate expression of an operably linked nucleic acid sequence.

To identify a modulator, one generally will determine the function of ATRS-regulated expression in the presence and absence of the candidate substance, a modulator defined as any substance that alters function. For example, a method generally comprises:

| | |
|---|---|
| (a) | providing a candidate modulator; |
| (b) | admixing the candidate modulator with an isolated compound or cell, |
| | or a suitable experimental animal; |
| (c) | measuring one or more characteristics of the compound, cell or |
| | animal in step (b); and |
| (d) | comparing the characteristic measured in step (c) with the character- |
| | istic of the compound, cell or animal in the absence of said candidate |
| | modulator, wherein a difference between the measured characteristics |
| | indicates that said candidate modulator is, indeed, a modulator of the |
| | compound, cell or animal. |

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them. Furthermore, a skilled artisan recognizes that any sequence which, analogous to ATRS, regulates expression of another may be tested in a similar fashion.

A. Modulators

As used herein the term “candidate substance,” or “agent” refers to any molecule that may potentially inhibit or enhance ATRS-regulated expression activity. The candidate substance may be a protein or fragment thereof, a small molecule, or even a nucleic acid molecule. It may prove to be the case that the most useful pharmacological compounds will be compounds that are structurally related to DNA binding molecules. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory or activating effect upstream, downstream or directly on the ATRS sequence. Regardless of the type of inhibitor or activator identified by the present screening methods, the effect of the inhibition or activator by such a compound results in affecting ATRS-regulated expresion as compared to that observed in the absence of the added candidate substance.

B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. In cyto Assays

The present invention also contemplates the screening of compounds for their ability to modulate ATRS-regulated expression in cells. Various cell lines can be utilized for such screening assays, including cells specifically engineered for this purpose. For example, a cell may preferably comprise a construct having the ATRS sequence operably linked to a reporter sequence. Assessment of a screened compound for affecting ATRS-regulated expression is based upon the effect it has on reporter sequence expression.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that interferes with ATRS-regulated expression. In these embodiments, the present invention is directed to a method for determining the ability of a candidate substance to upregulate or downregulate ATRS-regulated expression, generally including the steps of: administering a candidate substance to the animal; and determining the ability of the candidate substance to affect ATRS-regulated expression.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

VI. Cancer Therapies

A wide variety of cancer therapies, known to one of skill in the art, may be used in combination with the methods or compositions contemplated for the present invention. The inventors can use any of the treatments described herein in addition to administering to a cancer cell a construct comprising an ATRS sequence regulating expression of a therapeutic nucleic acid.

A. Radiotherapeutic Agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

B. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, surgery or sham surgery may be used in the model in the context of the present invention.

C. Chemotherapeutic Agents

These can be, for example, agents that directly cross-link DNA, agents that intercalate into DNA, and agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

D. Antibiotics

1. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

2. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

3. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways.

Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

4. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors which fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in.Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m^{2 , 450} mg/m², 475 mg/m², 500 mg/m². Of course, all of these dosages are exemplary, and any dosage in-between these points is also expected to be of use in the invention.

5. Bleomycin

Bleomycin is a mixture of cytotoxic glycopeptide antibiotics isolated from a strain of Streptomyces verticillus. It is freely soluble in water.

Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

Bleomycin may be given by the intramuscular, intravenous, or subcutaneous routes.

E. Miscellaneous Agents

1. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg//m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

In certain aspects of the current invention cisplatin is used in combination with emodin or emodin-like compounds in the treatment of non-small cell lung carcinoma. It is clear, however, that the combination of cisplatin and emodin and or emodin-like compounds could be used for the treatment of any other neu-mediated cancer.

2. VP16

VP16 is also know as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

3. Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

F. Plant Alkaloids

1. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also, expected to be of use in the invention.

2. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisolone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisolone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisolone. Vincristine is more useful than vinblastine in lymphocytic leukemia. Beneficial response have been reported in patients with a variety of other neoplasms, particularly Wilms' tumor, neuroblastoma, brain tumors, rhabdoniyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can alos be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

3. Vinblastine

When cells are incubated with vinblastine, dissolution of the microtubules occurs. Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours.

Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine for use will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

G. Alkylating Agents

1. Carmustine

Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisolone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m² 70 mg/m² 80 mg/m² 90 mg/m² 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject

2. Melphalan

Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKal of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

3. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

4. Chlorambucil

Chlorambucil (also known as leukeran) was first synthesized by Everett et al. (1953). It is a bifunctional alkylating agent of the nitrogen mustard type that has been found active against selected human neoplastic diseases. Chlorambucil is known chemically as 4-[bis(2-chlorethyl)amino] benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remingtons Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

5. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

6. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours.

The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

VII. Pharmaceutical Preparations

Pharmaceutical compositions of the present invention comprise an effective amount of one or more constructs comprising an ATRS sequence operably linked to a therapeutic nucleic acid sequence or additional agent dissolved or dispersed in a pharmaceutically acceptable carrier. The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. The preparation of a pharmaceutical composition that contains at least one construct comprising an ATRS sequence operably linked to a therapeutic nucleic acid sequence and/or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the composition is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations that are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

VIII. Nucleic Acid-Based Expression Systems

In some embodiments, the present invention regards a method comprising administration of a nucleic acid-based expression system, such as a vector comprising an ATRS sequence operably linked to a therapeutic polynucleotide. Generation of such vectors and any others useful for the practice of this invention are exemplified herein.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

1. Promoters and Enhancers

A skilled artisan recognizes that the ATRS sequence (such as SEQ ID NO:1 and SEQ ID NO:2) through which EGFR transcription activity acts may be used in conjunction with other regulatory sequences, such as promoters and/or enhancers. A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operably linked,” “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 1 lists non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/ | |
|---|---|
| Enhancer | References |
| Immunoglobulin | Banerji et al., 1983; Gilles et al., 1983; Grosschedl et |
| Heavy Chain | at., 1985; Atchinson et al., 1986, 1987; Imler et al., |
| | 1987; Weinberger et al., 1984; Kiledjian et al., 1988; |
| | Porton et al.; 1990 |
| Immunoglobulin | Queen et al., 1983; Picard et al., 1984 |
| Light Chain | |
| T-Cell Receptor | Luria et al., 1987; Winoto et al., 1989; Redondo et |
| | al.; 1990 |
| HLA DQ a and/ | Sullivan et al., 1987 |
| or DQ β | |
| β-Interferon | Goodbourn et al., 1986; Fujita et al., 1987; Goodbourn |
| | et al., 1988 |
| Interleukin-2 | Greene et al., 1989 |
| Interleukin-2 | Greene et al., 1989; Lib et al., 1990 |
| Receptor | |
| MHC Class II 5 | Koch et al., 1989 |
| MHC Class II | Sherman et al., 1989 |
| HLA-Dra | |
| β-Actin | Kawamoto et al., 1988; Ng et al.; 1989 |
| Muscle Creatine | Jaynes et al., 1988; Horlick et al., 1989; Johnson et |
| Kinase (MCK) | al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Ad- | Hirsch et al., 1990 |
| hesion Molecule | |
| (NCAM) | |
| α₁-Antitrypsin | Latimer et al., 1990 |
| H2B (TH2B) | Hwang et al., 1990 |
| Histone | |
| Mouse and/or | Ripe et al., 1989 |
| Type I Collagen | |
| Glucose-Regu- | Chang et al., 1989 |
| lated Proteins | |
| (GRP94 and | |
| GRP78) | |
| Rat Growth | Larsen et al., 1986 |
| Hormone | |
| Human Serum | Edbrooke et al., 1989 |
| Amyloid A | |
| (SAA) | |
| Troponin I (TN I) | Yutzey et al., 1989 |
| Platelet-Derived | Pech et al., 1989 |
| Growth Factor | |
| (PDGF) | |
| Duchenne Mus- | Klamut et al., 1990 |
| cular Dystrophy | |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen et |
| | al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky et |
| | al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al, 1988 |
| Human Immuno- | Muesing et al., 1987; Hauber et al., 1988; Jakobovits |
| deficiency Virus | et al., 1988; Feng et al., 1988; Takebe et al., 1988; |
| | Rosen et al., 1988; Berkhout et al., 1989; Laspia et al., |
| | 1989; Sharp et al., 1989; Braddock et al., 1989 |
| Cytomegalovirus | Weber et al., 1984; Boshart et al., 1985; Foecking et |
| (CMV) | al., 1986 |
| Gibbon Ape | Holbrook et al., 1987; Quinn et al., 1989 |
| Leukemia Virus | |

TABLE 2
Inducible Elements

| Element | Inducer | References |
|---|---|---|
| MT II | Phorbol Ester | Palmiter et al., 1982; Haslinger et |
| | (TFA) Heavy | al., 1985; Searle et al., 1985; |
| | metals | Stuart et al., 1985; Imagawa et |
| | | al., 1987, Karin et al., 1987; |
| | | Angel et al., 1987b; McNeall et |
| | | al., 1989 |
| MMTV (mouse | Glucocorticoids | Huang et al., 1981; Lee et al., |
| mammary tumor | | 1981; Majors et al., 1983; |
| virus) | | Chandler et al., 1983; Lee et al., |
| | | 1984; Ponta et al., 1985; Sakai et |
| | | al., 1988 |
| β-Interferon | Poly(rI)x | Tavernier et al., 1983 |
| | Poly(rc) | |
| Adenovirus 5 E2 | EIA | Imperiale et al., 1984 |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX Gene | Interferon, | Hug et al., 1988 |
| | Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2kb | | |
| HSP70 | EIA, SV40 | Taylor et al., 1989, 1990a, 1990b |
| | Large T | |
| | Antigen | |
| Proliferin | Phorbol Ester- | Mordacq et al., 1989 |
| | TPA | |
| Tumor Necrosis | PMA | Hensel et al., 1989 |
| Factor α | | |
| Thyroid Stimulating | Thyroid | Chatterjee et al., 1989 |
| Hormone α Gene | Hormone | |

The identity of tissue-specific promoters or elements, as well as assays to characterize their activity, is well known to those of skill in the art. Nonlimiting examples of such regions include the human LIMK2 gene (Nomoto et al 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al, 1998), DIA dopamine receptor gene (Lee, et al., 1997), insulin-like growth factor II (Wu et al., 1997), and human platelet endothelial cell adhesion molecule-I (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

9. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with P-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

10. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

a. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

b. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system for use in the compositions and/or methods of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al, 1988; McLaughlin et al., 1988). Details concerning the generation and use of rAAV vectors are described in U.S. Pat. Nos. 5,139,941 and 4,797,368, each incorporated herein by reference.

c. Retroviral Vectors

Retroviruses have promise as delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding a therapeutic sequence of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

d. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

e. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

B. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

1. Ex Vivo Transformation

Methods for tranfecting vascular cells and tissues removed from an organism in an ex vivo setting are known to those of skill in the art. For example, cannine endothelial cells have been genetically altered by retrovial gene tranfer in vitro and transplanted into a canine (Wilson et al., 1989). In another example, yucatan minipig endothelial cells were tranfected by retrovirus in vitro and transplated into an artery using a double-ballon catheter (Nabel et al., 1989). Thus, it is contemplated that cells or tissues may be removed and tranfected ex vivo using the nucleic acids of the present invention. In particular aspects, the transplanted cells or tissues may be placed into an organism. In preferred facets, a nucleic acid is expressed in the transplated cells or tissues.

2. Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985). The amount of composition used may vary upon the nature of the antigen as well as the organelle, cell, tissue or organism used

3. Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

To effect transformation by electroporation in cells such as, for example, plant cells, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species which have been transformed by electroporation of intact cells include maize (U.S. Pat. No. 5,384,253; Rhodes et al., 1995; D'Halluin et al., 1992), wheat (Zhou et al., 1993), tomato (Hou and Lin, 1996), soybean (Christou et al., 1987) and tobacco (Lee et al., 1989).

One also may employ protoplasts for electroporation transformation of plant cells (Bates, 1994; Lazzeri, 1995). For example, the generation of transgenic soybean plants by electroporation of cotyledon-derived protoplasts is described by Dhir and Widholm in International Patent Application No. WO 9217598, incorporated herein by reference. Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

4. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

5. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

6. Sonication Loading

Additional embodiments of the present invention include the introduction of a nucleic acid by direct sonic loading. LTK- fibroblasts have been transfected with the thymidine kinase gene by sonication loading (Fechheimer et al., 1987).

7. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

8. Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

9. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

C. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

A tissue may comprise a host cell or cells to be transformed with a construct such as one having an ATRS-regulated therapeutic nucleic acid sequence. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, anthers, ascite tissue, cobs, ears, flowers, husks, kernels, leaves, meristematic cells, pollen, root tips, roots, silk, stalks, and all cancers thereof.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art (see, for example, webpage http://phylogeny.arizona.edu/tree/phylogeny.html).

Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Cell types available for vector replication and/or expression include, but are not limited to, bacteria, such as E. coli (e.g., E. coli strain RR1, E. coli LE392, E. coli B, E. coli X 1776 (ATCC No. 31537) as well as E. coli W3110 (F-, lambda-, prototrophic, ATCC No. 273325), DH5α, JM109, and KC8, bacilli such as Bacillus subtilis; and other enterobacteriaceae such as Salmonella typhimurium, Serratia marcescens, various Pseudomonas specie, as well as a number of commercially available bacterial hosts such as SURE® Competent Cells and SOLOPACK™ Gold Cells (STRATAGENE®, La Jolla). In certain embodiments, bacterial cells such as E. coli LE392 are particularly contemplated as host cells for phage viruses.

Examples of eukaryotic host cells for replication and/or expression of a vector include, but are not limited to, HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

D. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. No. 5,871,986, 4,879,236, both herein incorporated by reference, and which can be bought, for example, under the name MAxBAC® 2.0 from INVITROGEN® and BACPACK™ BACULOVIRUS EXPRESSION SYSTEM FROM CLONTECH®.

Other examples of expression systems include STRATAGENE® 'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REx™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

It is contemplated that the proteins, polypeptides or peptides produced by the methods of the invention may be “overexpressed”, i.e., expressed in increased levels relative to its natural expression in cells. Such overexpression may be assessed by a variety of methods, including radio-labeling and/or protein purification. However, simple and direct methods are preferred, for example, those involving SDS/PAGE and protein staining or western blotting, followed by quantitative analyses, such as densitometric scanning of the resultant gel or blot. A specific increase in the level of the recombinant protein, polypeptide or peptide in comparison to the level in natural cells is indicative of overexpression, as is a relative abundance of the specific protein, polypeptides or peptides in relation to the other proteins produced by the host cell and, e.g., visible on a gel.

In some embodiments, the expressed proteinaceous sequence forms an inclusion body in the host cell, the host cells are lysed, for example, by disruption in a cell homogenizer, washed and/or centrifuged to separate the dense inclusion bodies and cell membranes from the soluble cell components. This centrifugation can be performed under conditions whereby the dense inclusion bodies are selectively enriched by incorporation of sugars, such as sucrose, into the buffer and centrifugation at a selective speed. Inclusion bodies may be solubilized in solutions containing high concentrations of urea (e.g. 8M) or chaotropic agents such as guanidine hydrochloride in the presence of reducing agents, such as β-mercaptoethanol or DTT (dithiothreitol), and refolded into a more desirable conformation, as would be known to one of ordinary skill in the art.

E. Proteins, Polypeptides, and Peptides

The present invention also provides purified, and in preferred embodiments, substantially purified, proteins, polypeptides, or peptides. The term “purified proteins, polypeptides, or peptides” as used herein, is intended to refer to an proteinaceous composition, isolatable from mammalian cells or recombinant host cells, wherein the at least one protein, polypeptide, or peptide is purified to any degree relative to its naturally-obtainable state, i.e., relative to its purity within a cellular extract. A purified protein, polypeptide, or peptide therefore also refers to a wild-type or mutant protein, polypeptide, or peptide free from the environment in which it naturally occurs.

The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or by any technique that would be know to those of ordinary skill in the art. Additionally, peptide sequences may be sythesized by methods known to those of ordinary skill in the art, such as peptide synthesis using automated peptide synthesis machines, such as those available from Applied Biosystems (Foster City, Calif.).

Generally, “purified” will refer to a specific protein, polypeptide, or peptide composition that has been subjected to fractionation to remove various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present invention, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

To purify a desired protein, polypeptide, or peptide a natural or recombinant composition comprising at least some specific proteins, polypeptides, or peptides will be subjected to fractionation to remove various other components from the composition. In addition to those techniques described in detail herein below, various other techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite, lectin affinity and other affinity chromatography steps; isoelectric focusing; gel electrophoresis; and combinations of such and other techniques.

Another example is the purification of a specific fusion protein using a specific binding partner. Such purification methods are routine in the art. As the present invention provides DNA sequences for the specific proteins, any fusion protein purification method can now be practiced. This is exemplified by the generation of an specific protein-glutathione S-transferase fusion protein, expression in E. coli, and isolation to homogeneity using affinity chromatography on glutathione-agarose or the generation of a polyhistidine tag on the N⁻ or C-terminus of the protein, and subsequent purification using Ni-affinity chromatography. However, given many DNA and proteins are known, or may be identified and amplified using the methods described herein, any purification method can now be employed.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Methods exhibiting a lower degree of relative purification may have advantages in total recovery of protein product, or in maintaining the activity of an expressed protein. Inactive products also have utility in certain embodiments, such as, e.g., in determining antigenicity via antibody generation.

### EXAMPLES

The following examples are offered by way of example, and are not intended to limit the scope of the invention in any manner.

### Example 1

### Nuclear EGFR in Highly Proliferative Tissues

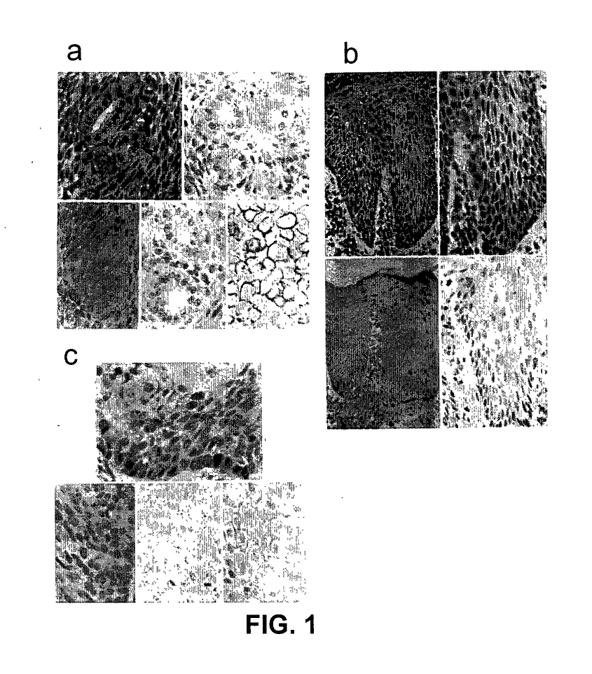

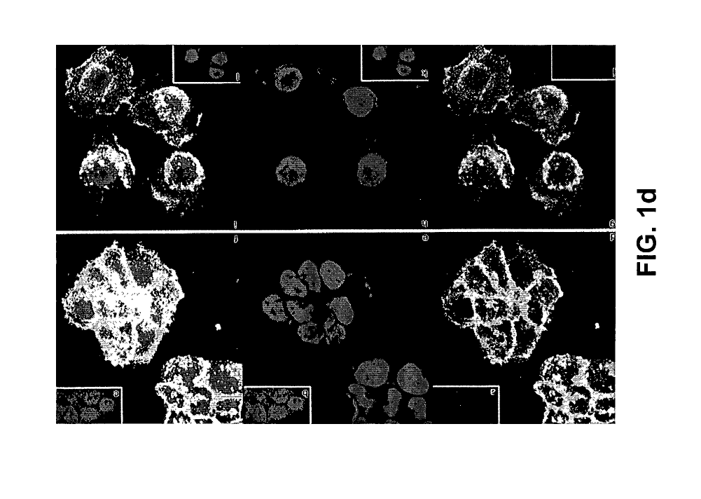

The localization of EGFR in the nucleus has been demonstrated (Zimmerman et al., 1995; Tervehauta et al., 1994; Kamio et al., 1990; Gusterson et al., 1985; Lipponene and Eskelinen, 1994). However, correlation between nuclear EGFR and the highly proliferating status of tissues was heretofore unknown. To further address this issue, EGFR expression in five different tissues was examined, including uterus from pregnant mice, mouse embryos, normal human mouth mucosa and different human cancer tissues. FIG. 1A (top, left) shows the immunostaining of EGF receptor on the uterus taken from a C3H-hen mouse on day 6 of pregnancy. The EGFR expression was high, and many of the cells showed the strong nuclear staining of the receptor. In contrast, EGFR was weakly expressed in uterus from non-pregnant mice, in which almost none of the cells showed the significant nuclear staining (FIG. 1A, top, right). The results of three control experiments shown in FIG. 1A (bottom) confirmed the specificity of EGFR signal. Heavy staining of nuclear EGFR was also observed in a 10-day-old of mouse embryo, which represents another example for the highly proliferating tissues. Next, samples were stained from human normal oral mucosa, which contained both highly proliferating basal cells and fully differentiated, not growing squamous cells. As a result, EGFR staining was detected only in the nuclei of the basal cells, as shown in FIG. 1B (top). The same tissue was stained with K₁67 antibody to confirm the correlation between nuclear receptor expression and the proliferating status of tissue (13.8% K₁67 positive for basal cells vs. 0.4% K₁67 positive for fully differentiated cells). Finally, human cancer tissue samples were examined, which is an important pathological example with highly proliferating activities. Although the membrane staining of EGFR was readily detectable (FIG. 1C, bottom, right), EGFR clearly localized in the nuclei in both the oral cancer (FIG. 1C, top) and the breast cancer samples. The similar nuclear staining of EGFR has also been clearly detected by using another EGFR antibody (Oncogene Science, EGF-R Ab1) against different epitopes, which further confirmed the specificity of the detected signals. Thus, in five different tissues with highly proliferating cells in vivo, clear nuclear EGFR staining was observed using different EGFR antibodies. Thus, there is a correlation between nuclear EGFR and high proliferation of tissues.

### Example 2

### Nuclear EGFR in in vitro Cultured Cell Lines

In addition to the tissue samples, EGFR-overexpressing cell lines were examined for the presence of nuclear EGFR. Since most EGFR is expressed on the cell surface in cell culture, which in turn may mask a weak EGFR signal in the nucleus, confocal microscopy was performed to assure the accuracy of the localization images. As shown in FIG. 1D, two EGFR overexpressors, A431 (top panel) and MDA-MB-468 (bottom panel) were immunostained with EGFR antibody (EGFR (1005)-G, Santa Cruz). Cells were double-labeled by fluorescein (FITC)-conjugated anti-EGFR antibody (1D, 1G panels) to localize EGFR (green signal) and by propidium iodide to localize nuclei (1E, 1H panels, red signal). When two images were merged, a significant portion of EGFR was localized in the nucleus (1F, 1I panels, yellow signal). The same procedure was also performed by using FITC-conjugated preimmune serum as a negative control (1A, 1J panels). Nuclear staining was readily detectable by propidium iodide (1B, 1K panels), however, no yellow signal was detectable when two images were merged (1C, 1L panels), assuring the specificity of the nuclear EGFR detected in panels 1F and 1I. The EGFR signal observed was specific because nuclear EGFR was also detected when two other antibodies recognizing different epitopes were used (EGF-R Ab-1, Oncogene Science; EGFR Ab-3, NeoMarkers).

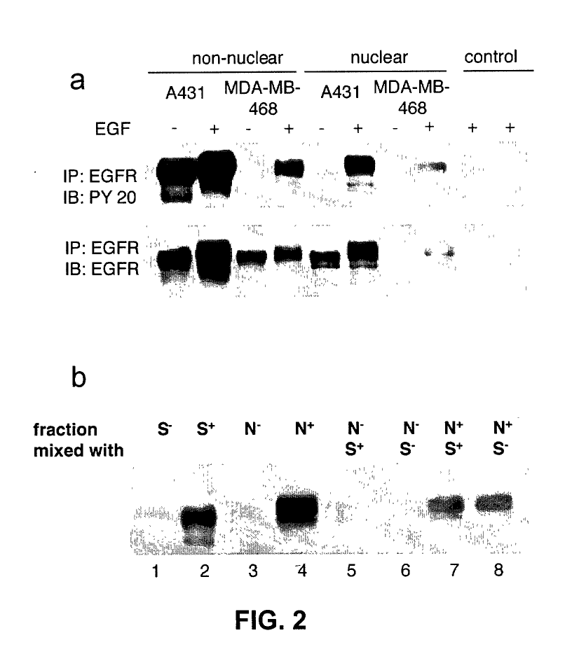

To further confirm the presence of EGFR in the nuclear fraction and to determine the phosphorylation status of the nuclear receptor, nuclear extracts from A431 and MDA-MB-468 cells were subjected to immunoprecipitation with anti-EGFR antibody followed by immunoblotting with either anti-phosphotyrosine antibody (FIG. 2A, top) or anti-EGFR antibody (FIG. 2A, bottom). Nuclear EGFR levels increased upon treatment with EGF and EGFR, which accumulated in the nucleus and was highly tyrosine-phosphorylated. Similar results were also obtained when cells were treated with another ligand for EGFR, TGFA.

To rule out the possibility that the signal seen in the nuclear extracts was due to contamination, the nuclei from unstimulated and EGF-stimulated MDA-MB-468 cells (N⁻ and N⁺, respectively) were mixed with the non-nuclear fraction from the cells unstimulated (S⁻) or stimulated with EGF for 30 minutes (S⁺). The nuclei were then separated from the non-nuclear fraction again, washed extensively and the nuclear extract was then analyzed by immunoblotting with anti-phosphotyrosine antibody. In this way, it was demonstrated that even if the non-nuclear fraction of the EGF-activated cell lysate was mixed and incubated for 30 minutes with nuclei from the untreated cells, the EGFR in that fraction did not diffuse into or contaminate the nuclear fraction (FIG. 2B, lane 5). Thus, the nuclear EGFR that was detected was not due to passive contamination of the nuclear fraction by EGFR from the plasma membrane or cytoplasm. This experiment also demonstrated that the cell-surface and cytoplasmic EGFR did not passively pass into the nuclei but indicated the existence of a specific pathway for transporting the receptor into the nuclei. This pathway was evidently not intact when the cells were disrupted as there was failure of the isolated nuclei to uptake EGFR from the non-nuclear fraction.

### Example 3

### Nuclear EGFR is from the Cell Surface

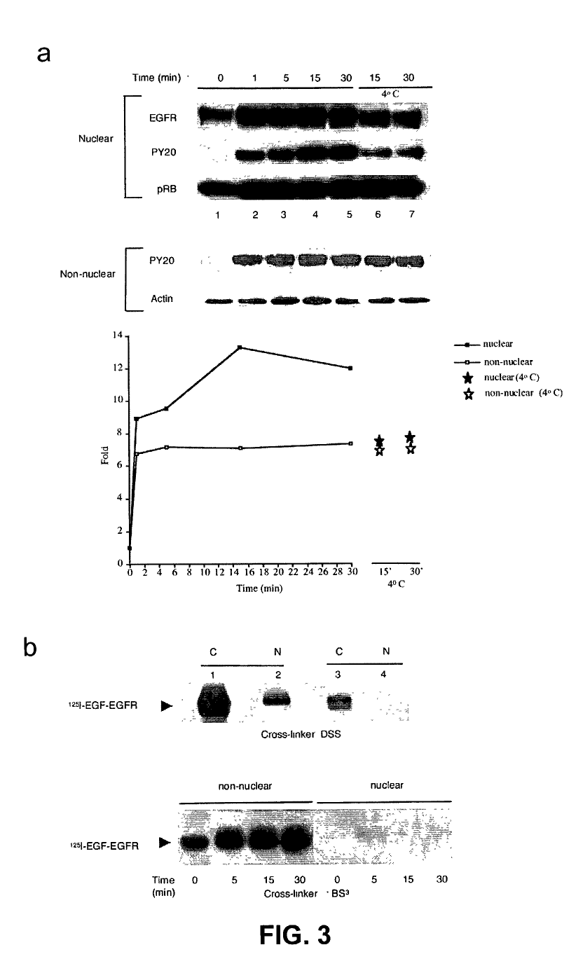

Having established a putative pathway for the nuclear import of EGFR, the kinetics and source of the translocation of nuclear EGFR was characterized. To determine the kinetics of the translocation, A431 cells were incubated with EGF for 1 to 30 minutes. FIG. 3A (top) shows that phosphorylated nuclear EGFR was detected as quick as 1 minute after EGF treatment, with the peak detected at about 15-30 minutes. This rapid translocation was also seen in MDA-MB-468 cells. However, when cells were treated with EGF for one minute and immediately replaced with cold medium to stop membrane trafficking, the translocation of the receptor was abolished even though cells were continuously incubated in the medium with the same concentration of ligand (lanes 6 and 7).

In contrast, the level of phosphorylated EGFR in the non-nuclear fraction remained the same in this time period (FIG. 3A, middle). Also, stopping the membrane trafficking did not affect the EGFR phosphorylation in the non-nuclear fraction (lanes 6 and 7). These results not only confirmed that the increase of the phosphorylated EGFR in nuclear extract was not due to contamination by the non-nuclear fraction, but also suggested that the nuclear receptor may have come from the cell membrane (FIG. 3A, bottom panel).

To further confirm this possibility, ¹²⁵I-labeled EGF was crosslinked to EGFR on the cell surface of MDA-MB-468 cells by means of the non-cleavable, amine reactive homobifunctional cross-linker disuccinimidyl suberate (DSS) (Pilch and Czech, 1979) (FIG. 3B, top) or a non-cleavable, membrane-impermeable cross-linking reagent, bis(sulfosuccinimidyl) suberate (BS³) (FIG. 3B, bottom). As shown in FIG. 3B, (top), a clear cross-linked ¹²⁵I-EGF-EGFR band was detected in the nuclear extract (lane 2). This band could be competed away in the presence of an excess of cold EGF (lane 4), thus confirming the specificity of the binding. As shown in FIG. 3B (bottom), the movement of ¹²⁵I-EGF-EGFR complex could be detected in the nuclear extract as early as 5 minutes after cross-linking. Since BS³ could not pass through cell membrane alone, the detection of the 125I-EGF-EGFR complex confirmed that EGFR was moving into the nuclei from the cell surface after EGF stimulation.

### Example 4

### A Strong Transactivation Domain in EGFR

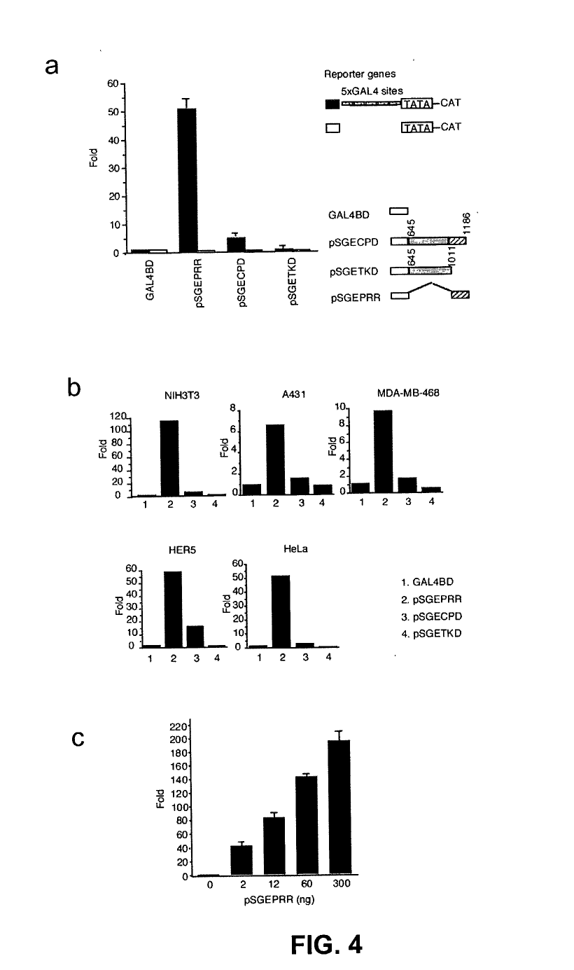

Having characterized the time- and ligand-dependent nuclear translocation of EGFR, the receptor's biological function(s) in the nucleus was examined. Previous studies had already shown that EGF and EGFR both formed complexes with chromatin, especially in transcriptionally active regions (Rakowicz-Szulczynska et al., 1986), thus suggesting that EGFR and its ligand may play roles in modulating gene expression. Therefore, specific domains of EGFR that exhibited transactivation activity were sought. By motif analysis, it was found that the C-terminus of EGFR contained a proline-rich sequence, which was a typical feature of transactivation domain for transcription factors. When the C-terminus of EGFR (PSEGPRR) was fused to the GAL4 DNA-binding domain, this chimeric construct strongly activated (up to 60-fold) the transcription of a reporter gene containing five GAL4 binding sites linked to chloramphenicol transferase (CAT) cDNA in NIH 3T3 cells (FIG. 4A). In contrast, the tyrosine kinase domain and the whole cytoplasmic domain of EGFR either did not activate (tyrosine kinase domain) or only slightly activated (whole cytoplasmic domain) the transcription of the reporter.

The observed transactivating activity was evidently dependent on DNA binding since the C-terminus of EGFR failed to activate the reporter gene when the GAL4 binding sites were deleted. This activity was also evidently general, and not cell-type specific, because it occurred in all five cell lines tested (FIG. 4B). Finally, this transactivation activity was dose-dependent; by increasing the amount of expression vector, a very nice dose-dependent activation was observed (FIG. 4C).

The weaker transactivation activity of the whole cytoplasmic domain suggested the presence of a negative control activity in the tyrosine kinase domain. It is interesting to note that the tyrosine kinase domain contains the negative regulatory sites identified by other groups in the early studies (Khazaie et al., 1993).

### Example 5

### DNA Binding Site(s) for EGFR Complexes

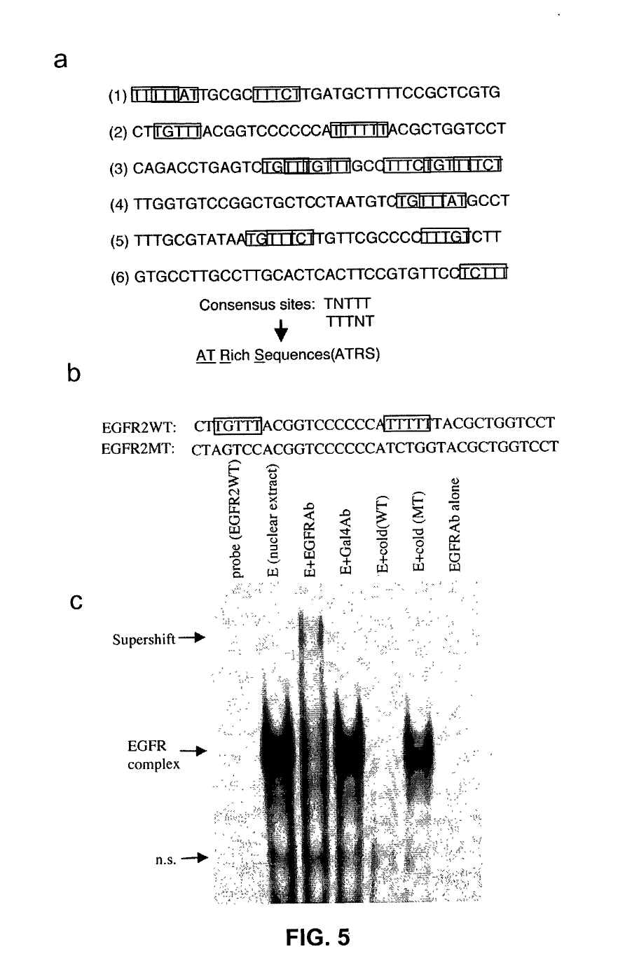

As a putative transcription factor, DNA binding of EGFR was tested. Upon a positive result, the DNA binding site(s) by cyclic amplification and selection of targets (CASTing) method (Wright et al., 1991) was identified. In brief, cell lysate prepared from EGF-treated A431 cells as the source of EGFR was incubated with an excess amount of oligonucleotides containing a 36-nucleotide core of random sequences flanked by two PCR primers (see Example 8). To identify the EGFR contained DNA binding complexes, EGFR antibody (Ab12, NeoMarkers) was added into the reaction mixture to supershift the complexes of interest. A very faint band appeared after the antibody was added. This band contained EGFR because a supershift was detected only by EGFR antibody but not by the control GAL4 antibody or mouse preimmune serum (see FIG. 5B). In addition, the EGFR antibody alone did not bind to the probe when the cell lysate was absent in the reaction. After excision of the band, the DNA was eluted and amplified by PCR. The same procedure was repeated for another three rounds. The final DNA products were then subcloned and sequenced. When the sequences were compared from all six clones, an AT-rich minimal consensus sequence (ATRS) was identified that appeared in all six clones for eighteen times in total. The first consensus ATRS sequence is TNTTT (SEQ ID NO:1) and the second consensus sequence is TTTNT (SEQ ID NO:2). Thus, this consensus sequence is an EGFR-associated sequence (FIG. 5A).

Next, using one of the cloned sequences as the probe, gel retardation assays were performed to confirm the specific association between EGFR and the identified sequences. As expected, specific binding of an EGFR-containing complex to the probe was observed, which could be competed away by an excess of cold wild-type oligonucleotides but not by the oligonucleotides in which the consensus sites were mutated (FIG. 5B). Therefore, the ATRS is a true DNA binding site for the EGFR complex.

### Example 6

### EGFR-Binding-Site Dependent Gene Activation

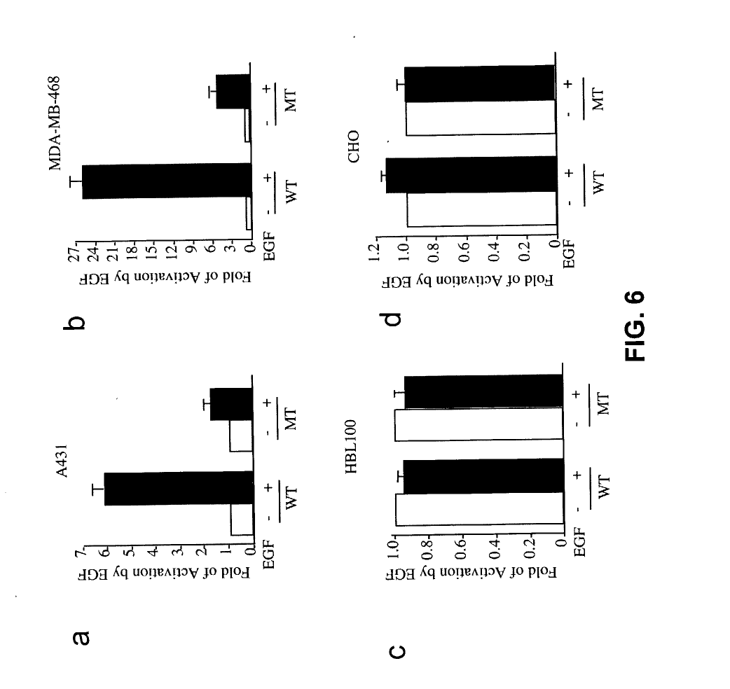

Next, four repeats of either the ATRS sites or the mutated sites were constructed in a luciferase reporter construct containing the mouse thymidine kinase gene (TK) minimal promoter. When the reporter genes were transfected into EGFR-overexpressing cell lines (A431 and MDA-MB-468), EGF could strongly activate the wild-type reporter construct but only weakly activate the mutant construct (FIG. 6A and 6B). In contrast, neither wild type nor the mutant reporter could be activated by EGF in the cells with low (HBL100) or no EGFR (CHO) (FIGS. 6C and 6D). EGFR-dependent activation, however, could be restored in these two cell lines upon the cotransfection of the EGFR expression vector. Thus, the ATRS, which can bind to nuclear EGFR complex, responds to EGFR-dependent activation, whereas the mutant ATRS, which fails to bind to nuclear EGFR, loses it ability to respond to the EGFR-dependent activation. This result indicates that ATRS is a target sequence(s) activated by nuclear EGFR.

### Example 7

### Cyclin D1 as one of the Potential Targets for Nuclear EGFR

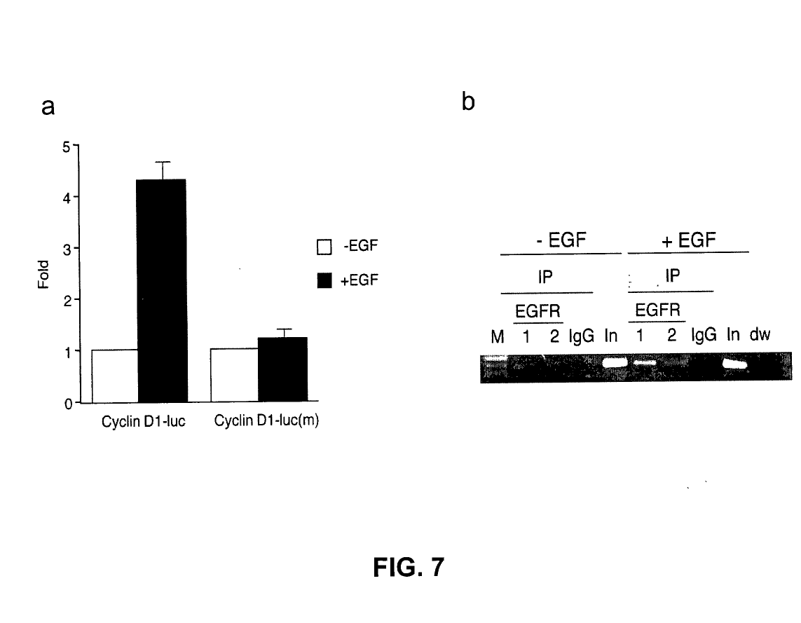

Targets for nuclear EGFR were identified. Since nuclear EGFR correlated with highly proliferative activity of cells, it was suspected that its target genes were likely involved in cell proliferation. One of the candidates, Cyclin D1, is a cell-cycle regulator essential for G1 phase progression. Overexpression of Cyclin D1 has been shown to shorten G1 phase and accelerate cell proliferation. Two ATRS sequences are located in the proximal region of Cyclin D1 promoter between nucleotide −74 to −70 (TTTAT; SEQ ID NO:3) and −31 to −27 (TTTGT; SEQ ID NO:4), respectively. To test whether these two ATRS in Cyclin D1 promoter were responsive to EGFR activity, the reporter gene containing 163 bp of the Cyclin Dl promoter (Cyclin D1-luc) was tested. Many known transcription factor-binding sites had been eliminated from this construct but it still contained two ATRS described above. As shown in FIG. 7A, EGF activated wild-type reporter up to four fold but the activation was abolished when these two ATRS sequences were mutated [Cyclin D1-luc(m)], in which the two ATRS were changed to CCTAT (SEQ ID NO:5) and GGTGT (SEQ ID NO:6), respectively.

Furthermore, to examine whether EGFR can directly bind to promoter region of Cyclin D1 in vivo, chromatin immunoprecipitation assays were performed using EGFR antibodies to precipitate EGFR with or without EGF (100 ng/ml) stimulation. As shown in FIG. 7B, the EGFR was found physically associated with promoter region of Cyclin D1 only in EGF-treated cell extracts precipitated by EGFR-specific antibodies but not by normal IgG. These data indicate that EGFR can bind to Cyclin D1 promoter in vi vo.

### Example 8

### Significance of the Present Invention

hi the Examples herein, it is demonstrated that EGFR shares several features with transcription factors: it can be located in the nucleus, contains a transactivation domain, associates with genes, and activates sequence-specific gene expression. Therefore, the results indicate nuclear EGFR functions as a transcription factor. The data presented herein is the first study to demonstrate that EGFR can bind to specific DNA sequences to activate gene expression. The demonstration of Cyclin D1, a well known cell growth promoting gene, as its potential target explains why nuclear localization of EGFR was strongly correlated with the tissues with highly proliferation activity.

Given that rat SDGF, a ligand for EGFR, can bind to AT-rich DNA sequences that perfectly match the ATRS and must be transported into nucleus to induce a mitogenic response (Kimura, 1993), in a specific embodiment EGFR and its ligands function together as transactivation complexes in which the ligand serves as the DNA binding domain, and the receptor as the transactivation domain.

EGFR contains a nuclear localization signal (NLS) in amino acid residues 645-657 of the cytoplasmic domain (RRRHIVRKRTLRR; SEQ ID NO:7); when fused to this polypeptide, β-galactosidase could be directed into nucleus. Therefore, in a specific embodiment, EGFR is translocated into the nucleus through the conventional nuclear importing system associated with the nuclear pore complex (i.e., the Ran/Importin pathway).

Other transmembrane receptors have also been detected in the nucleus (Jans and Hassan, 1998), including the receptors for insulin (Vigneri et al., 1978), nerve growth factor (Rakowicz-Szulczynska et al., 1986; Rakowicz-Szulczynska et al., 1988) fibroblast growth factor (Maher, 1996; Stachowizk et al., 1996), platelet-derived growth factor (Rakowicz-Szulczynska et al., 1986), growth hormone (Lobie et al., 1994), IL-1 Curtis et al., 1990), c-erbB-4 (Srinivasan et al., 2000) and HER-2/neu (Xie and Hung, 1994; Cohen et al., 1992). However, the functions of transmembrane receptors in the nucleus have never been elucidated. In contrast, the function of nuclear EGFR to act as a transactivator for a specific target gene, particularly in proliferating tissue, indicates that EGFR function and/or its binding to a target sequence are useful objectives for therapeutic potential in highly proliferative tissues.

### Example 9

### Methods

Cell culture and nuclear fractionation. All cell lines were normally grown in DMEMIF-12 with 10% fetal calf serum. Before EGF stimulation, cells were serum-starved for 24 hours. Then, cells were stimulated with EGF (100 ng/ml) for different time periods. Cells were then lysed in a lysis buffer (20 mM HEPES, pH 7.0, 10 mM potassium chloride, 2 mM magnesium chloride, 0.5% NP-40, 1 mM sodium vanadate, 1 mM PMSF, 0.15 U/ml aprotinin) and homogenized in a tight-fitting Dounce homogenizer by 30 strokes. The homogenate was centrifuged at 1500× g for 5 minutes to sediment the nuclei. The supernatant was then resedimented at 15,000× g for 5 minutes, and the resulting supernatant was used as the “non-nuclear fraction”. The nuclear pellet was washed three times and resuspended in the same buffer containing 0.5 M NaCl to extract nuclear proteins. The extracted material was sedimented at 15,000× g for 10 minutes and the resulting supernatant was termed the “nuclear fraction”.

Immunohistochemical staining. Immunostaining was done using a modification of the avidin-biotin complex technique described previously (Hsu et al., 1981). The Confocal Microscope used in this analysis was Multiprobe 2001 Inverted CLSM system, Molecular Dynamics (Sunnyvale, Calif.).

Western blotting and immunoprecipitation. Cellular extract immunoprecipitated by anti-human EGFR monoclonal antibodies (Amersham and NeoMarkers) was separated by SDS-PAGE and transferred onto a nitrocellulose membrane. Immunoblotting was performed with anti-EGFR antibody (UBI and NeoMarkers) or anti-phosphotyrosine monoclonal antibody (mAb2; Oncogene Science) as primary antibody followed by horseradish peroxidase-conjugated rabbit anti-sheep or anti-mouse antibodies as secondary antibodies and detected by chemoluminescence (ECL, Amersham).

The kinetics study of the EGFR nuclear localization. A431 cells were first stimulated with EGF (100 ng/ml). They were then incubated at 37° C. for 1-30 minutes or incubated at 37° C. for 1 minute and then switched to cold medium containing the same concentration of EGF for another 14 or 29 minutes. Then, the nuclear and the non-nuclear fractions were isolated and subjected to western blotting with anti-EGFR or anti-phosphotyrosine (PY20) antibody on nitrocellulose membranes. The same membranes were also probed with pRB (for the nuclear fraction) or actin (for the non-nuclear fraction) as a loading control.

Chemical cross-linking. MDA-MB-468 cells were grown in low-serum media for 24 hours. The plates were cooled to 4° C. before adding media containing ¹²⁵I-EGF (1 μCi/ml) and further incubating for 20 min. Phosphate buffer containing BS³ (3 mM) was then added to the plates and incubated with gentle rocking for 30 min on ice. The crosslinking was quenched by washing cells with cold buffer containing 25 mM Tris, pH 7.4, and 140 mM NaCl. This was followed by washing with 50 mM glycine-hydrochloride, pH 3.0, 150 mM NaCl to remove excess non-cross-linked ¹²⁵I-EGF. After further washing with cold PBS, warm media was replaced onto the cells and the cells were incubated at 37° C. for a further 15 min to allow cross-linked proteins to enter cells. The cells were then lysed as before, and nuclear and non-nuclear fractions were subjected to 8% SDS-PAGE.

Plasmids and transfection. The cytoplasmic domain (645-1186), tyrosine kinase domain (645-1011), and C-terminal proline rich region (1011-1086) of human EGFR were amplified by PCR and subcloned in frame into pSG424 to generate for each domain a fusion protein containing GAL4 DNA binding proteins. The fusion protein constructs were then transfected into a variety of cell lines along with GAL4CAT or GAL4Luc reporter plasmids via liposomal DC-Cole provide by Dr. Leaf Huang at University of Pittsburgh.

CASTing and EMSA. Cyclic amplification and selection of targets (CASTing) was performed as described previously (Wright et al., 1991). Briefly, a 76-bp oligonucleotide containing a random stretch of 36 nucleotides (5′-GACGTCTCGAGAATTCATCG(N)₃₆CGATGGATCCATCCATGTCAGACT-3′; SEQ ID NO:8), a 5′-end PCR primer (5′-GACGTCTCGAGAATTCATCG-3′; SEQ ID NO:9) and a 3′-end PCR primer CGATGGATCCATCCATGTCAGACT-3′; SEQ ID NO:10) were synthesized. Double-stranded DNA was generated and ³²P labeled by PCR as the probe. A431 cell lysate was incubated with EGFR antibody (Ab12, NeoMarkers) in a buffer containing 25 mM HEPES, 100 mM KCl, 0.5 mM MgCl₂, 1 mg/ml bovine serum albumin, 10% glycerol, 5 mM dithiothreitol, and 0.1 mg poly(dI-dC) on ice for 30 minutes. Then, the probe was added to the reaction and incubated at room temperature for another 30 minutes prior to electrophoresis on a 4% nondenaturing polyacrylamide gel. The shifted band was excised, and bound DNAs were eluted. The cluted DNAs were then amplified and labeled with PCR by using the two primers described above. After four rounds of selection, the amplified products were subcloned into pBluescript and sequenced. For electrophoretic mobility shift assay (EMSA): A431 nuclear extract was dialyzed with the binding buffer first and then incubated with EGFR or control antibody in the same buffer on ice for 30 minutes. Then, EMSAs were performed exactly as described above.

Chromatin Immunoprecipitation assay (CHIP). The methods of Braunstein et al. (1993) and Orlando (Orlando and Paro, 1993) were adopted as follows. A431 cells of 5 dishes of 10 cm dish were serum starved for 24 hr and stimulated with EGF (100 ng/ml) for 30 min. The cells were then treated with formaldehyde (1% final concentration) for 10 min to crosslink proteins to DNA before harvesting. Cells were scraped off the plate, washed with ice-cold phosphate-buffered saline and resuspended in 500 μl of hypotonic buffer [10 mM Tris-HCl, pH 7.4, 10 mM KCL, 1 mM dithiothreitol (DTT)], and passed 20 times through a 25 gauge needle. Nuclei were spun down, resuspended in 200 μl SDS lysis buffer (1% SDS, 10 mM EDTA, 50 mM Tris-HCl, pH 8.0 and protease inhibitors), and sonicated for two 30 s bursts separated by cooling on ice. After centrifugation, the supernatant was diluted 10-fold with immunoprecipitation buffer (50 mM Tris-HCl, pH 8.0, 150 mM NaCl, 5 mM EDTA, 0.5% NP40). The cell lysate were precleared by incubation at 4° C. for 1 hr with normal rabbit IgG and for another 1 hr with protein A agarose beads. The cleared lysates were incubated with two different anti-EGFR antibodies (Santa Cruz or NeoMarkers), or normal rabbit IgG at 4° C. overnight. Immunoprecipitated complexes were collected by adding protein A agarose beads for 2 hr at 4° C. Immunoprecipitates were washed once with RIPA (150 mM NaCl, 50 mM Tris-HCl, pH 8.0, 0.1% SDS, 0.5% sodium deoxylcholate, 1.0% NP40), once in high salt wash (500 mM NaCl, 1.0% NP40, 0.1% SDS, 50 mM Tris-HCl, pH 8.0), once in LiCl wash (250 mM LiCl, 1.0% NP40, 0.5% sodium deoxylcholate, 1 mM EDTA, 50 mM Tris-HCl, pH 8.0) and twice in TE buffer (10 mM Tris-HCl, pH 8.0, 1 mM EDTA). The beads were then treated with RNase (50 μg/ml) for 30 min at 37° C. The samples were adjusted to 0.25% SDS, 250 μg/ml proteinase K and incubated at 37° C. overnight. The cross-links were reversed by heating at 65° C. for 6 hr and the DNA was then extracted with phenol/chloroform and was ethanol-precipitated. Specific sequences of cyclin D1 promoter in the immunoprecipitates were detected by PCR using the following primers: S (5′-GAGGGGACTAATATTTCCAGCAA-3′; SEQ ID NO:11) and AS (5′-TAAAGGGATTTCAGCTTAGCA-3′; SEQ ID NO:12).

One skilled in the art readily appreciates that the patent invention is well adapted to carry out the objectives and obtain the ends and advantages mentioned as well as those inherent therein. Methods, compositions, sequences, plasmids, vectors, pharmaceutical compositions, treatments, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

### Example 10

### Therapeutic Agents

The ATRS sequence (using, for example, SEQ ID NO:1 or SEQ ID NO:2) operably linked to a therapeutic polynucleotide as it relates to its anti-tumor activity is tested in an animal study. The ATRS sequence operably linked to a therapeutic polynucleotide is delivered by a vector, such as a liposome or adenoviral vector, into nude mice models for its anti-tumor activity. Once the anti-tumor activity is demonstrated, potential toxicity is further examined using immunocompetent mice, followed by clinical trials.

In a specific embodiment, the preferential growth inhibitory activity of ATRS sequence operably linked to a therapeutic polynucleotide is tested in animal. Briefly, EGFR-overexpressing cancer cell lines are administered into mouse cancer model. After the tumors reach a particular size, the ATRS sequence operably linked to a therapeutic polynucleotide or control is intravenously injected into the mouse in an admixture with an acceptable carrier, such as liposomes. The tumor sizes and survival curve from these treatments are compared and statistically analyzed. In a preferred embodiment, the ATRS sequence operably linked to a therapeutic polynucleotide is better and preferentially inhibits the growth of tumor compared to that of the control.

### Example 11

### Clinical Trials

This example is concerned with the development of human treatment protocols using the ATRS sequence (such as SEQ ID NO:1 or SEQ ID NO:2) operably linked to a therapeutic polynucleotide alone or in combination with other anti-cancer drugs. The ATRS sequence operably linked to a therapeutic polynucleotide and anti-cancer drug treatment will be of use in the clinical treatment of various cancers. Such treatment will be particularly useful tools in anti-tumor therapy, for example, in treating patients with breast cancer, glioblastoma, head and neck cancer, bladder cancer, pancreatic cancer, colon cancer, lung cancer, thyroid cancer, and/or brain cancer that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the breast cancer, glioblastoma, head and neck cancer, bladder cancer, pancreatic cancer, colon cancer, lung cancer, thyroid cancer, and/or brain cancer in clinical trials.

Patients with advanced metastatic breast cancer, glioblastoma, head and neck cancer, bladder cancer, pancreatic cancer, colon cancer, lung cancer, thyroid cancer, and/or brain cancer or other cancers chosen for clinical study will typically be at high risk for developing the cancer, will have been treated previously for the cancer which is presently in remission, or will have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the ATRS sequence operably linked to a therapeutic polynucleotide and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), Akt (phosphorylated and un-phosphorylated forms) and in the cells (ATRS sequence operably linked to a therapeutic polynucleotide) may be assessed and recorded.

In the same procedure, the ATRS sequence operably linked to a therapeutic polynucleotide may be administered alone or in combination with the other anti-cancer drug. The administration may be in the pleural/peritoneal cavity, directly into the tumor, or in a systemic manner. The starting dose may be 0.5 mg/kg body weight. Three patients may be treated at each dose level in the absence of grade>3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the ATRS sequence operably linked to a therapeutic polynucleotide, and the lot of anti-cancer drug exceed 5 EU/kg for any given patient.

The the ATRS sequence operably linked to a therapeutic polynucleotide and/or the other anti-cancer drug combination, may be administered over a short infusion time or at a steady rate of infusion over a 7 to 21 day period. The ATRS sequence operably linked to a therapeutic polynucleotide infusion may be administered alone or in combination with the anti-cancer drug and/or emodin like tyrosine kinase inhibitor. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the ATRS sequence operably linked to a therapeutic polynucleotide in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phopshorylated forms), EGFR expression, Akt (phosphorylated and non-phosphorylated forms), p185, etc.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, ki67 and Tunel assay to measure apoptosis, Akt) and in the cells (Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. An urinalysis may be performed every 4 weeks.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

### PATENTS

U.S. Pat. No. 4,684,611

U.S. Pat. No. 4,797,368

U.S. Pat. No. 4,952,500

U.S. Pat. No. 5,139,941

U.S. Pat. No. 5,302,523

U.S. Pat. No. 5,322,783

U.S. Pat. No. 5,384,253

U.S. Pat. No. 5,464,765

U.S. Pat. No. 5,538,877

U.S. Pat. No. 5,538,880

U.S. Pat. No. 5,550,318

U.S. Pat. No. 5,563,055

U.S. Pat. No. 5,580,859

U.S. Pat. No. 5,589,466

U.S. Pat. No. 5,591,616

U.S. Pat. No. 5,610,042

U.S. Pat. No. 5,656,610

U.S. Pat. No. 5,736,524

U.S. Pat. No. 5,702,932

U.S. Pat. No. 5,780,448

U.S. Pat. No. 5,789,215

U.S. Pat. No. 5,945,100

U.S. Pat. No. 5,981,274

U.S. Pat. No. 5,994,136

U.S. Pat. No. 5,994,624

U.S. Pat. No. 6,013,516

EPO 0273085

PCT Application No. WO 92/17598

PCT Application WO 94/09699

PCT Application WO 95/06128

### PUBLICATIONS

Almendro N, Bellon T, Rius C, Lastres P, et al. Cloning of the human platelet endothelial cell adhesion molecule-1 promoter and its tissue-specific expression. Structural and functional characterization. J Immunol Dec. 15, 1996;157(12):5411-21.

Anderson, D. et al. Binding of SH2 domains of phospholipase C gamma 1, GAP, and Src to activated growth factor receptors. Science 250, 979-82. (1990).

Angel et al., Cell, 49:729, 1987b.

Angel et al., Mol. Cell. Biol., 7:2256, 1987a.

Atchison and Perry, Cell, 46:253, 1986.

Atchison and Perry, Cell, 48:121, 1987.

Banerji et al., Cell, 27:299, 1981.

Banerji et al., Cell, 35:729, 1983.

Berkhout et al., Cell, 59:273, 1989.

Blanar et al., EMBO J., 8:1139, 1989.

Bodine and Ley, EMBO J., 6:2997, 1987.

Boonstra, J. et al. The epidermal growth factor. Cell Biol Int 19, 413-30. (1995).

Boshart et al., Cell, 41:521, 1985.

Bosze et al., EMBO J., 5:1615, 1986.

Braddock et al., Cell, 58:269, 1989.

Braunstein, M., Rose, A. B., Holmes, S. G., Allis, C. D. & Broach, J. R. Transcriptional silencing in yeast is associated with reduced nucleosome acetylation. Genes Dev 7, 592-604. (1993).

Bulla and Siddiqui, J. Virol., 62:1437, 1986.

Campbell and Villarreal, Mol. Cell. Biol., 8:1993, 1988.

Campere and Tilghman, Genes and Dev., 3:537, 1989.

Campo et al., Nature, 303:77, 1983.

Carpenter, G. & Cohen, S. Epidermal growth factor. Annu Rev Biochem 48, 193-216 (1979).

Celander and Haseltine, J. Virology, 61:269, 1987.

Celander et al., J. Virology, 62:1314, 1988.

Chandler et al., Cell, 33:489, 1983.

Chang et al., Mol. Cell. Biol., 9:2153, 1989.

Chatterjee et al., Proc. Nat'l Acad. Sci. USA., 86:9114, 1989.

Choi et al., Cell, 53:519, 1988.

Cohen et al., “A Repetitive Sequence Element 3′ of the human c-Ha-rasl Gene Has Enhancer Activity,” J. Cell. Physiol., 5:75, 1987.

Cohen, J. A., Yachnis, A. T., Arai, M., Davis, J. G. & Scherer, S. S. Expression of the neu proto-oncogene by Schwann cells during peripheral nerve development and Wallerian degeneration. J Neurosci Res 31, 622-34. (1992).

Cohen, S., Ushiro, H., Stoscheck, C. & Chinkers, M. A native 170,000 epidermal growth factor receptor-kinase complex from shed plasma membrane vesicles. J Biol Chem 257, 1523-31. (1982).

Costa et al., Mol. Cell. Biol., 8:81, 1988.

Cripe et al., EMBO J., 6:3745, 1987.

Culotta and Hamer, Mol. Cell. Biol., 9:1376, 1989.

Curtis, B. M., Widmer, M. B., deRoos, P. & Qwamstrom, E. E. IL-1 and its receptor are translocated to the nucleus. J Immunol 144, 1295-303. (1990).

Dandolo et al., J. Virology, 47:55, 1983.

De Villiers et al., Nature, 312:242, 1984.

Defize, L. H., Moolenaar, W. H., van der Saag, P. T. & de Laat, S. W. Dissociation of cellular responses to epidermal growth factor using anti-receptor monoclonal antibodies. Embo J 5, 1187-92. (1986).

Deschamps et al., Science, 230:1174, 1985.

Edbrooke et al., Mol. Cell. Biol., 9:1908, 1989.

Edlund et al., Science, 230:912, 1985.

Feng and Holland, Nature, 334:6178, 1988.

Firak and Subramanian, Mol. Cell. Biol., 6:3667, 1986.

Foecking and Hofstetter, “Powerful and/or Versatile Enhancer-Promoter Unit for [mammalian, plant, fungus, bacteria?] Expression Vectors,” Gene, 45:101, 1986.

Fujita et al., Cell, 49:357, 1987.

Gilles et al., Cell, 33:717, 1983.

Gloss et al., EMBO J., 6:3735, 1987.

Godbout et al., Mol. Cell. Biol., 8:1169, 1988.

Goodboum and Maniatis, Proc. Nat'l Acad. Sci. USA, 85:1447, 1988.

Goodboum et al., Cell, 45:601, 1986.

Greene et al., Immunology Today, 10:272, 1989.

Grosschedl and Baltimore, Cell, 41:885, 1985.

Gusterson, B. et al. Evidence for increased epidermal growth factor receptors in human sarcomas. Int J Cancer 36, 689-93. (1985).

Haslinger and Karin, Proc. Nat'l Acad. Sci. USA., 82:8572, 1985.

Hauber and Cullen, J. Virology, 62:673, 1988.

Hen et al., Nature, 321:249, 1986.

Hensel et al., Lymphokine Res., 8:347, 1989.

Herr and Clarke, Cell, 45:461, 1986.

Hirochika et al., J. Virol., 61:2599, 1987.

Hirsch et al., Mol. Cell. Biol., 10:1959, 1990.

Holbrook et al., Virology, 157:211, 1987.

Horlick and Benfield, Mol. Cell. Biol., 9:2396, 1989.

Hsu, S. M., Raine, L. & Fanger, H. Use of avidin-biotin-peroxidase complex (ABC) in immunoperoxidase techniques: a comparison between ABC and unlabeled antibody (PAP) procedures. J Histochem Cytochem 29, 577-80. (1981).

Hu, P. et al. Interaction of phosphatidylinositol 3-kinase-associated p85 with epidermal growth factor and platelet-derived growth factor receptors. Mol Cell Biol 12, 981-90. (1992). Huang et al., Cell, 27:245, 1981.

Hug H, Costas M, Staeheli P, Aebi M, et al. Organization of the murine Mx gene and characterization of its interferon- and virus-inducible promoter. Mol Cell Biol Aug. 8, 1998 (8):3065-79.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol., 7:2558, 1987.

Jakobovits et al., Mol. Cell. Biol., 8:2555, 1988.

Jameel and Siddiqui, Mol. Cell. Biol., 6:710, 1986.

Jans, D. A. & Hassan, G. Nuclear targeting by growth factors, cytokines, and their receptors: a role in signaling? Bioessays 20, 400-11. (1998).

Jaynes et al., Mol. Cell. Biol., 8:62, 1988.

Johnson et al., Mol. Cell. Biol., 9:3393, 1989.

Kadesch and Berg, Mol. Cell. Biol., 6:2593, 1986.

Kamio, T., Shigematsu, K., Sou, H., Kawai, K. & Tsuchiyama, H. Immunohistochemical expression of epidermal growth factor receptors in human adrenocortical carcinoma. Hum Pathol 21, 277-82. (1990).

Karin et al., Mol. Cell. Biol., 7:606, 1987.

Katinka et al., Cell, 20:393, 1980.

Katinka et al., Nature, 290:720, 1981.

Kawamoto et al., Mol. Cell. Biol., 8:267, 1988.

Khazaie, K., Schirrmacher, V. & Lichtner, R. B. EGF receptor in neoplasia and metastasis. Cancer Metastasis Rev 12, 255-74. (1993).

Kiledjian et al., Mol. Cell. Biol., 8:145, 1988.

Kimura, H. Schwannoma-derived growth factor must be transported into the nucleus to exert its mitogenic activity. Proc Natl Acad Sci U S A 90, 2165-9. (1993).

Klamut et al., Mol. Cell. Biol., 10:193, 1990.

Knauer, D. J., Wiley, H. S. & Cunningham, D. D. Relationship between epidermal growth factor receptor occupancy and mitogenic response. Quantitative analysis using a steady state model system. J Biol Chem 259, 5623-31. (1984).

Koch et al., Mol. Cell. Biol., 9:303, 1989.

Kraus J, Woltje M, Schonwetter N, Hollt V. Alternative promoter usage and tissue specific expression of the mouse somatostatin receptor 2 gene. FEBS Lett May 29, 1998; 428(3): 165-70.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984a.

Kriegler et al., Cell, 53:45, 1988.

Kriegler et al., In: Cancer Cells 2/Oncogenes and Viral Genes, Van de Woude et al. eds, Cold Spring Harbor, Cold Spring Harbor Laboratory, 1984b.

Kriegler et al., In: Gene Expression, D. Hamer and M. Rosenberg, eds., New York: Alan R. Liss, 1983.

Kuhl et al., Cell, 50:1057, 1987.

Kunzetal., Nucl. Acids Res., 17:1121, 1989.

Lareyre J J, Thomas T Z, Zheng W L, Kasper S, et al. A 5-kilobase pair promoter fragment of the murine epididymal retinoic acid-binding protein gene drives the tissue-specific, cell-specific, and androgen-regulated expression of a foreign gene in the epididymis of transgenic mice. J Biol Chem Mar. 19, 1999;274(12):8282-90.

Larsen et al., Proc. Nat'l Acad. Sci. USA., 83:8283, 1986.

Laspia et al., Cell, 59:283, 1989.

Latimer et al., Mol. Cell. Biol., 10:760, 1990.

Lee et al., Mol. Endocrinol., 2: 404-411, 1988.

Lee et al., Nature, 294:228, 1981.

Lee S H, Wang W, Yajima S, Jose P A, et al. Tissue-specific promoter usage in the D1A dopamine receptor gene in brain and kidney. DNA Cell Biol Nov. 16, 1997 (11):1267-75.

Levinson et al., Nature, 295:79, 1982.

Lin et al., Mol. Cell. Biol., 10:850, 1990.

Lipponen, P. & Eskelinen, M. Expression of epidermal growth factor receptor in bladder cancer as related to established prognostic factors, oncoprotein (c-erbB-2, p53) expression and long-term prognosis. Br J Cancer 69, 1120-5. (1994).

Lobie, P. E., Wood, T. J., Chen, C. M., Waters, M. J. & Norstedt, G. Nuclear translocation and anchorage of the growth hormone receptor. J Biol Chem 269, 31735-46. (1994).

Luria et al., EMBO J., 6:3307, 1987.

Lusky and Botchan, Proc. Nat'l Acad. Sci. USA., 83:3609, 1986.

Lusky et al., Mol. Cell. Biol., 3:1108, 1983.

Maher, P. A. Nuclear Translocation of fibroblast growth factor (FGF) receptors in response to FGF-2. J Cell Biol 134, 529-36. (1996).

Majors and Varmus, Proc. Nat'l Acad. Sci. USA., 80:5866, 1983.

Marti, U. and Wells, A., Mol. Cell Biol. Res. Commun., 3,8-14, 2000.

Marti, U. et al., Thyroid, 11(2), 137-145,2001.

McNeall et al., Gene, 76:81, 1989.

Miksicek et al., Cell, 46:203, 1986.

Mordacq and Linzer, Genes and Dev., 3:760, 1989.

Moreau et al., Nucl. Acids Res., 9:6047, 1981.

Muesing et al., Cell, 48:691, 1987.

Ng et al., Nuc. Acids Res., 17:601, 1989.

Nomoto S, Tatematsu Y, Takahashi T, Osada H. Cloning and characterization of the alternative promoter regions of the human LIMK2 gene responsible for alternative transcripts with tissue-specific expression. Gene Aug. 20, 1999;236(2):259-71.

Ondek et al., EMBO J., 6:1017, 1987.

Orlando, V. & Paro, R. Mapping Polycomb-repressed domains in the bithorax complex using in vivo formaldehyde cross-linked chromatin. Cell 75, 1187-98. (1993).

Ornitz et al., Mol. Cell. Biol., 7:3466, 1987.

Palmiter et al., Nature, 300:611, 1982.

Pech et al., Mol. Cell. Biol., 9:396, 1989.

Perez-Stable and Constantini, Mol. Cell. Biol., 10:1116, 1990.

Picard and Schaffner, Nature, 307:83, 1984.

Pilch, P. F. & Czech, M. P. Interaction of cross-linking agents with the insulin effector system of isolated fat cells. Covalent linkage of 125I-insulin to a plasma membrane receptor protein of 140,000 daltons. J Biol Chem 254, 3375-81. (1979).

Pinkert et al., Genes and Dev., 1:268, 1987.

Ponta et al., Proc. Nat'l Acad. Sci. USA., 82:1020, 1985.

Porton et al., Mol. Cell. Biol., 10:1076, 1990.

Queen and Baltimore, Cell, 35:741, 1983.

Quinn et al., Mol. Cell. Biol., 9:4713, 1989.

Rakowicz-Szulczynska, E. M., Herlyn, M. & Koprowski, H. Nerve growth factor receptors in chromatin of melanoma cells, proliferating melanocytes, and colorectal carcinoma cells in vitro. Cancer Res 48, 7200-6. (1988).

Rakowicz-Szulczynska, E. M., Rodeck, U., Herlyn, M. & Koprowski, H. Chromatin binding of epidermal growth factor, nerve growth factor, and platelet-derived growth factor in cells bearing the appropriate surface receptors. Proc Natl Acad Sci U S A 83, 3728-32. (1986).

Redondo et al., Science, 247:1225, 1990.

Reisman and Rotter, Mol. Cell. Biol., 9:3571, 1989.

Remington's Pharmaceutical Sciences, 18th Ed. Mack Printing Company, 1990.

Resendez Jr. et al., Mol. Cell. Biol., 8:4579, 1988.

Ripe et al., Mol. Cell. Biol., 9:2224, 1989.

Rittling et al., Nucl. Acids Res., 17:1619, 1989.

Rosen et al., Cell, 41:813, 1988.

Satake et al., “Biological Activities of Oligonucleotides Spanning the F9 Point Mutation Within the Enhancer Region of Polyoma Virus DNA,” J. Virology, 62:970, 1988.

Schaffner et al., J. Mol. Biol., 201:81, 1988.

Searle et al., Mol. Cell. Biol., 5:1480, 1985.

Sharp and Marciniak, Cell, 59:229, 1989.

Shaul and Ben-Levy, EMBO J., 6:1913, 1987.

Sherman et al., Mol. Cell. Biol., 9:50, 1989.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Srinivasan, R., Gillett, C. E., Bames, D. M. & Gullick, W. J. Nuclear expression of the c-erbB-4/HER-4 growth factor receptor in invasive breast cancers. Cancer Res 60, 1483-7. (2000).

Stachowiak, M. K., Maher, P. A., Joy, A., Mordechai, E. & Stachowiak, E. K. Nuclear accumulation of fibroblast growth factor receptors is regulated by multiple signals in adrenal medullary cells. Mol Biol Cell 7, 1299-317. (1996).

Stephens and Hentschel, Biochem. J., 248:1, 1987.

Stuart et al., Nature, 317:828, 1985.

Sullivan and Peterlin, Mol. Cell. Biol., 7:3315, 1987.

Swartzendruber and Lehman, J. Cell. Physiology, 85:179, 1975.

Takebe et al., Mol. Cell. Biol., 8:466, 1988.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Tervahauta, A., Syijanen, S. & Sy janen, K. Epidermal growth factor receptor, c-erbB-2 proto-oncogene and estrogen receptor expression in human papillomavirus lesions of the uterine cervix. Int J Gynecol Pathol 13, 234-40. (1994).

Thiesen et al., J. Virology, 62:614, 1988.

Treisman, Cell, 42:889, 1985.

Tronche et al., Mol. Biol. Med., 7:173, 1990.

Tronche et al., Mol. Cell. Biol., 9:4759, 1989.

Trudel and Constantini, Genes and Dev., 6:954, 1987.

Tsumaki N, Kimura T, Tanaka K, Kimura J H, et al. Modular arrangement of cartilage- and neural tissue-specific cis-elements in the mouse alpha2(XI) collagen promoter. J Biol Chem Sep. 4, 1998;273(36):22861-4.

Tyndall et al., Nuc. Acids. Res., 9:6231, 1981.

Vannice and Levinson, J. Virology, 62:1305, 1988.

Vasseur et al., Proc. Nat'l Acad. Sci. USA., 77:1068, 1980.

Vigneri, R., Goldfine, I. D., Wong, K. Y., Smith, G. J. & Pezzino, V. The nuclear envelope. The major site of insulin binding in rat liver nuclei. J Biol Chem 253, 2098-103. (1978).

Wakshull, E. M. & Wharton, W. Stabilized complexes of epidermal growth factor and its receptor on the cell surface stimulate RNA synthesis but not mitogenesis. Proc Natl Acad Sci U S A 82, 8513-7. (1985).

Wang and Calame, Cell, 47:241, 1986.

Weber et al., Cell, 36:983, 1984.

Weinberger et al. Mol. Cell. Biol., 8:988, 1984.

Winoto and Baltimore, Cell, 59:649, 1989.

Wright, W. E., Binder, M. & Funk, W. Cyclic amplification and selection of targets (CASTing) for the myogenin consensus binding site. Mol Cell Biol 11, 4104-10. (1991).

Wu H K, Squire J A, Song Q, Weksberg R. Promoter-dependent tissue-specific expressive nature of imprinting gene, insulin-like growth factor II, in human tissues. Biochem Biophys Res Commun Apr. 7, 1997;233(1):221-6.

Xie, Y. & Hung, M. C. Nuclear localization of p185^{neu} tyrosine kinase and its association with transcriptional transactivation. Biochem Biophys Res Commun 203, 1589-98. (1994).

Yutzey et al. “An Internal Regulatory Element Controls Troponin I Gene Expression,” Mol. Cell. Biol., 9:1397, 1989.

Yutzey et al. Mol. Cell. Biol., 9:1397, 1989.

Zhao-Emonet J C, Boyer O, Cohen J L, Klatzmann D. Deletional and mutational analyses of the human CD4 gene promoter: characterization of a minimal tissue-specific promoter. Biochim Biophys Acta Nov. 8, 1998;1442(2-3):109-19.

Zimmermann, H. et al. The overexpression of proliferating cell nuclear antigen in biliary cirrhosis in the rat and its relationship with epidermal growth factor receptor. J Hepatol 23, 459-64. (1995).

We claim:

1. A method of treating a cell having upregulated EGFR expression, comprising administering to said cell a nucleic acid sequence comprising an EGFR-regulated promoter sequence operably linked to a therapeutic polynucleotide.

2. The method of claim 1, wherein the EGFR-regulated promoter sequence is SEQ ID NO:1 or SEQ ID NO:2.

3. The method of claim 1, wherein said nucleic acid sequence comprises multiple copies of said EGFR-regulated promoter sequence operably linked to said therapeutic polynucleotide.

4. The method of claim 1, wherein said therapeutic polynucleotide is a tumor suppressor, tumor associated gene, growth factor, growth-factor receptor, signal transducer, hormone, cell cycle regulator, nuclear factor, transcription factor or apoptic factor.

5. The method of claim 4, wherein said tumor suppressor is selected from the group consisting of Rb, p53, p16, p19, p21, p73, DCC, APC, NF-1, NF-2, PTEN, FHIT, C-CAM, E-cadherin, MEN-I, MEN-II, ZACI, VHL, FCC, MCC, PMS1, PMS2, MLH-1, MSH-2, DPC4, BRCA1, BRCA2 and WT-1.

6. The method of claim 4, wherein said growth-factor receptor is selected from the group consisting of FMS, ERBB/HER, ERBB-2/NEU/HER-2, ERBA, TGF-β receptor, PDGF receptor, MET, KIT and TRK.

7. The method of claim 4, wherein said signal transducer is selected from the group consisting of SRC, AB1, RAS, AKT/PKB, RSK-1, RSK-2, RSK-3, RSK-B, PRAD, LCK and ATM.

8. The method of claim 4, wherein said transcription factor or nuclear factor is selected from the group consisting of JUN, FOS, MYC, BRCA1, BRCA2, ERBA, ETS, EVII, MYB, HMGI-C, HMGI/LIM, SKI, VHL, WT1, CEBP-a, NFKB, IKB, GLI and REL.

9. The method of claim 4, wherein said growth factor is selected from the group consisting of SIS, HST, INT-1/WT1 and INT-2.

10. The method of claim 4, wherein said apoptic factor is selected from the group consisting of Bax, Bak, Bim, Bik, Bid, Bad, Bcl-2, Harakiri, granzyme B and ICE proteases.

11. The method of claim 4, wherein said tumor associated gene is selected from the group consisting of CEA, mucin, MAGE and GAGE.

12. The method of claim 1, wherein said cell is in vivo.

13. The method of claim 1, wherein said cell is in an individual having a proliferative disorder.

14. The method of claim 13, wherein said proliferative disorder is cancer.

15. The method of claim 12, wherein said cell is in a human.

16. The method of claim 1, wherein said cell is in vitro.

17. A method of screening for a modulator of an EGFR-regulated promoter sequence, comprising:
introducing to a cell a nucleic acid construct comprising a nucleic acid sequence having at least one copy of the EGFR-regulated promoter sequence operably linked to a reporter sequence;
contacting the cell with a candidate modulator; and
assaying for a change in expression of said reporter sequence.

18. The method of claim 17, wherein the EGFR-regulated promoter sequence is SEQ ID NO:1 or SEQ ID NO:2.

19. The method of claim 17, wherein said reporter sequence expression is upregulated.

20. The method of claim 17, wherein said reporter sequence expression is downregulated.

21. The method of claim 17, wherein said reporter sequence is selected from the group consisting of luciferase, green fluorescent protein, blue fluorescent protein, β-galactosidase, and chloramphenicol acetyl transferase.

22. The method of claim 17, wherein said candidate modulator is a protein, a small molecule, a nucleic acid molecule, an antisense molecule, a ribozyme, an antibody, or a combination thereof.

23. The method of claim 17, wherein said candidate modulator is determined to be a modulator of an EGFR-regulated promoter sequence.

24. The method of claim 23, further comprising the step of administering to an individual with cancer a pharmaceutically acceptable formulation of said modulator.

25. A method for identifying transcription factor activity for a receptor tyrosine kinase, comprising assaying said receptor tyrosine kinase for DNA binding activity.

26. The method of claim 25, further comprising identifying the target DNA sequence of said DNA binding.

27. The method of claim 25, wherein said receptor tyrosine kinase is selected from the group consisting of insulin receptor, nerve growth factor receptor, fibroblast growth factor receptor, platelet-derived growth factor receptor, growth hormone receptor, IL-1 receptor, HER/neu, interferon alpha receptor, interferon beta receptor, and interferon gamma receptor, IL-5 receptor, angiogenin receptor, erythropoietin receptor, and G-CSF (granulocyte colony stimulating factor) receptor.

28. The method of claim 25, wherein said DNA binding activity of said receptor tyrosine kinase is direct.

29. The method of claim 25, wherein said DNA binding activity of said receptor tyrosine kinase is through an agent that binds the target directly.

30. A method of treating cancer in an individual comprising the step of reducing translocation of a receptor tyrosine kinase from a membrane of a cancerous cell of said individual to the nucleus of said cell.

31. The method of claim 30, wherein said receptor tyrosine kinase is EGFR.

32. A method of treating cancer in an individual comprising the step of reducing transcription factor activity of a receptor tyrosine kinase in a cancerous cell of said individual.

33. The method of claim 32, wherein said receptor tyrosine kinase is EGFR.

34. As a composition of matter, a pharmaceutical composition comprising:
a nucleic acid construct comprising a nucleic acid sequence of SEQ ID NO:1 or SEQ ID NO:2 operably linked to a therapeutic nucleic acid sequence; and
a pharmaceutically acceptable carrier.

35. The composition of matter of claim 34, wherein said therapeutic nucleic acid sequence is a tumor suppressor, tumor associated gene, growth factor, growth-factor receptor, signal transducer, hormone, cell cycle regulator, nuclear factor, transcription factor or apoptic factor.

36. A method of identifying a cancerous cell in an individual, comprising identifying a nuclearly localized receptor tyrosine kinase in said cell.

37. The method of claim 36, wherein the receptor tyrosine kinase is EGFR.

38. The method of claim 36, wherein said cancerous cell is a breast cancer cell, glioblastoma cell, head and neck cancer cell, bladder cancer cell, pancreatic cancer cell, colon cancer cell, lung cancer cell, thyroid cancer cell, or brain cancer cell.

39. The method of claim 36, further comprising the step of treating said individual for said cancer.

40. The method of claim 39, wherein said treating step comprises administering to said individual a pharmaceutically acceptable formulation of a nucleic acid sequence comprising an EGFR-regulated promoter sequence operably linked to a therapeutic polynucleotide.

41. The method of claim 40, wherein the EGFR-regulated promoter sequence is SEQ ID NO:1 or SEQ ID NO:2.

42. The method of claim 39, wherein said treating step comprises administering to said individual a pharmaceutically acceptable formulation of a modulator that inhibits transcriptional activity of a receptor tyrosine kinase.

43. A method of treating an individual with cancer comprising administering to said individual a modulator that affects EGFR transcriptional activity.
